(19) United States
(12) Patent Application Publication
(10) Pub. No.: US 2006/0069084 A1
(43) Pub. Date: Mar. 30, 2006

(54) Methods of inhibiting kinases

(76) Inventors: Christopher John Burns, Melbourne (AU); Andrew Frederick Wilks, South Yarra (AU)

Correspondence Address: MORRISON & FOERSTER LLP, 12531 HIGH BLUFF DRIVE, SUITE 100, SAN DIEGO, CA, 92130-2040, US

(21) Appl. No.: 11/223,633

(22) Filed: Sep. 9, 2005

Related U.S. Application Data
(62) Division of application No. 10/470,955, filed on Jul. 30, 2003.

(30) Foreign Application Priority Data
Jan. 30, 2001 (AU) PR2792
Jan. 30, 2001 (AU) PR2793

(51) Int. Cl. A61K 31/551; A61K 31/5377; A61K 31/498

(52) U.S. Cl. 514/218; 514/234.2; 514/249

(57) ABSTRACT

The present invention provides methods of inhibiting JAK involving the use of a group of compounds based either upon a 2-amino-6-carba-disubstituted pyrazine scaffold or a 2-amino-6-carba-disubstituted pyridine scaffold. The invention also provides methods of treating JAK-associated disease states.

### CROSS-REFERENCE TO RELATED APPLICATION

This application is a divisional of U.S. application Ser. No. 10/470,955, filed Jul. 30, 2003, which is the national phase of PCT Application PCT/AU2/00089 having an international filing date of 30 Jan. 2002, and claims priority from Australian Application Nos. PR2792 and PR2793 filed 30 Jan. 2001. The contents of each of these documents are incorporated herein by reference in their entirety.

### FIELD OF THE INVENTION

The present invention relates generally to the field of non-peptidyl inhibitors of protein tyrosine kinases. More particularly, the present invention concerns methods of inhibiting specific protein tyrosine kinases, including members of the JAK family of protein tyrosine kinases.

### BACKGROUND OF THE INVENTION

Since the immune system is central to the protection of an individual from an external biological threat, diseases of the immune system are therefore a consequence of one or a combination of three problems with the immune system:

Underproduction or suppression of the immune system (e.g. AIDS or SIDS);

Overproduction of cells of the immune system (e.g Leukemia or Lymphoma);

Overproduction of the effects of the immune system (e.g. Inflammation); and

Inappropriate activation of the effects of the immune system (e.g. allergy).

Treatments of diseases of the immune system are therefore aimed at either the augmentation of immune response or the suppression of inappropriate responses. Since cytokines play a pivotal role in the regulation of the immune system, they are appropriately considered to be key targets for therapeutic intervention in immune pathologies. Similarly, the intracellular signal transduction pathways that are regulated by cytokines are potential points of therapeutic intervention in diseases that involve overproduction of cytokine signalling.

There are many different types of protein kinases. Each type has the ability to add a phosphate group to an amino acid in a target protein. The phosphate is provided by hydrolyzing ATP to ADP. Typically, a protein kinase has an ATP-binding site and a catalytic domain that can bind to the target protein molecule. The JAK family of protein tyrosine kinases (PTKs) play a central role in the cytokine dependent regulation of the proliferation and end function of several important cell types of the immune system.

A direct comparison of the four currently known mammalian JAK family members reveals the presence of seven highly conserved domains (Harpur et al, 1992). In seeking a nomenclature for the highly conserved domains characteristic of this family of PTKs, the classification used was guided by the approach of Pawson and co-workers (Sadovski et al, 1986) in their treatment of the SRC homology (SH) domains. The domains have been enumerated accordingly with most C-terminal homology domain designated JAK Homology domain 1 (JH1). The next domain N-terminal to JH1 is the kinase-related domain, designated here as the JH2 domain. Each domain is then enumerated up to the JH7 located at the N-terminus. The high degree of conservation of these JAK homology (JH) domains suggests that they are each likely to play an important role in the cellular processes in which these proteins operate. However, the boundaries of the JAK homology domains are arbitrary, and may or may not define functional domains. Nonetheless, their delineation is a useful device to aid the consideration of the overall structural similarity of this class of proteins.

The feature most characteristic of the JAK family of PTKs is the possession of two kinase-related domains (JH1 and JH2) (Wilks et al, 1991). The putative PTK domain of JAK1 (JH1) contains highly conserved motifs typical of PTK domains, including the presence of a tyrosine residue at position 1022 located 11 residues C-terminal to sub-domain VII that is considered diagnostic of membership of the tyrosine-specific class of protein kinases. Alignment of the human JAK1 PTK domain (255 amino acids), with other members of the PTK class of proteins revealed homology with other functional PTKs (for example, 28% identity with c-fes (Wilks and Kurban, 1988) and 37% homology to TRK (Kozma et al, 1988). The JH1 domains of each of the JAK family members possess a interesting idiosyncrasy within the highly conserved sub-domain VIII motif (residues 1015 to 1027 in JAK2) that is believed to lie close to the active site, and define substrate specificity. The phenylalanine and tyrosine residues flanking the conserved tryptophan in this motif are unique to the JAK family of PTKs. Aside from this element, the JH1 domains of each of the members of the JAK family are typical PTK domains.

The central role played by the JAK family of protein tyrosine kinases in the cytokine dependent regulation of the proliferation and end function of several important cell types means that agents which inhibit JAK are useful in the prevention and chemotherapy of disease states dependent on these enzymes. Potent and specific inhibitors of each of the currently known four JAK family members will provide a means of inhibiting the action of those cytokines that drive immune pathologies, such as asthma (e.g. IL-13; JAK1, JAK2), and leukemia/lymphoma (e.g. IL-2: JAK1 and JAK3).

Furthermore, certain types of cancer such as prostate cancer develop autocrine production of certain cytokines as a selectable mechanism of developing growth and/or metastatic potential. An example of this is cancer of the prostate, where IL-6 is produced by and stimulates the growth of prostate cancer cell lines such as TSU and TC3 (Spiotto M T, and Chung T D, 2000). Interestingly, levels of IL-6 are elevated in sera of patients with metastatic prostate cancer.

A great deal of literature covers the area of cytokine signalling. The present inventors have focussed on the JAK/STAT pathway that is involved in the direct connection of cytokine receptor to target genes (such as cell cycle regulators (e.g. p21) and anti-apoptosis genes (such as Bcl-X_{L})). The JAK/STAT Pathway

The delineation of a particularly elegant signal transduction pathway downstream of the non-protein tyrosine kinase cytokine receptors has recently been achieved. In this pathway the key components are: (i) A cytokine receptor chain (or chains) such as the Interleukin-4 receptor or the Interferon y receptor; (ii) a member (or members) of the JAK family of PTKs; (iii) a member(s) of the STAT family of transcription factors, and (iv) a sequence specific DNA element to which the activated STAT will bind.

A review of the JAK/STAT literature offers strong support to the notion that this pathway is important for the recruitment and marshalling of the host immune response to environmental insults, such as viral and bacterial infection. This is well exemplified in Table 1 and Table 2. Information accumulated from gene knock-out experiments have underlined the importance of members of the JAK family to the intracellular signalling triggered by a number of important immune regulatory cytokines. The therapeutic possibilities stemming from inhibiting (or enhancing) the JAK/STAT pathway are thus largely in the sphere of immune modulation, and as such are likely to be promising drugs for the treatment of a range of pathologies in this area. In addition to the diseases listed in Tables 1 and 2, inhibitors of JAKs could be used as immunosuppresive agents for organ transplants and autoimmune diseases such as lupus, multiple sclerosis, rheumatoid arthritis, Type I diabetes, autoimmune thyroid disorders, Alzheimer's disease and other autoimmune diseases. Additionally, treatment of cancers such as prostate cancer by JAK inhibitors is indicated.

TABLE 1

| | Cell Types | |
|---|---|---|
| Disease Type | Involved | Characteristics |
| Atopy | | |
| Allergic Asthma | (Mast Cells) | T-cell activation of B-cells |
| Atopic Dermatitis (Eczema) | (Eosinophils) | followed by IgE mediated |
| Allergic Rhinitis | (T-Cells) | activation of resident Mast |
| | (B-Cells) | cells and Eosinophils |
| Cell Mediated | | |
| Hypersensitivity | | |
| Allergic Contact Dermatitis | (T-cells) | T-cell hypersensitivity |
| Hypersensitivity | (B-cells) | |
| Pneumonitis | | |
| Rheumatic Diseases | | |
| Systemic Lupus | (Monocytes) | Cytokine Production |
| Erythematosus (SLE) | (Macrophages) | (e.g.TNF, IL-1, CSF-1, |
| Rheumatoid Arthritis | (Neutrophils) | GM-CSF) |
| Juvenile Arthritis | (Mast Cells) | T-cell Activation |
| Sjögren's Syndrome | (Eosinophils) | JAK/STAT activation |
| Scleroderma | (T-Cells) | |
| Polymyositis | (B-Cells) | |
| Ankylosing Spondylitis | | |
| Psoriatic Arthritis | | |
| Viral Diseases | | |
| Epstein Barr Virus (EBV) | Lymphocytes | JAK/STAT Activation |
| Hepatitis B | Hepatocytes | JAK/STAT Activation |
| Hepatitis C | Hepatocytes | JAK/STAT Inhibition |
| HIV | Lymphocytes | JAK/STAT Activation |
| HTLV 1 | Lymphocytes | JAK/STAT Activation |
| Varicella-Zoster Virus | Fibroblasts | JAK/STAT Inhibition |
| (VZV) | | |
| Human Papilloma Virus | Epithelial cells | JAK/STAT Inhibition |
| (HPV) | | |
| Cancer | | |
| Leukemia | Leucocytes | Cytokine production |
| Lymphoma | Lymphocytes | JAK/STAT Activation |

TABLE 2
Diseases Potentially Treatable By JAK-Based Drug Therapies

| | | JAK family | Strength of |
|---|---|---|---|
| Target Disease | Cytokine | member | Association |
| Asthma | IL-4 & IL-9 | JAK1 &JAK3 | +++ |
| | IL-13 | JAK1 & JAK2 | +++ |
| | IL-5 | JAK2 | +++ |
| Eczema | IL-4 | JAK1 & JAK3 | +++ |
| | IFN-α | JAK1 & JAK2 | +++ |
| Food Allergy | IL-4 | JAK1 & JAK3 | +++ |
| Inflammatory Bowel | IL-4 | JAK1 & JAK3 | +++ |
| Disease & Crohn's | | | |
| Disease | | | |
| Leukaemia And | (IL-2) | JAK3, JAK1 & | +++ |
| Lymphoma | | JAK2 | |
| Cutaneous | GM-CSF & | JAK1 & JAK2 | +++ |
| Inflammation | IL-6 | | |
| Immune Suppression | IL-10 | JAK1 & TYK2 | +++ |
| By Solid Tumour | | | |
| Prostate Cancer | IL-6 | JAK1, JAK2 | +++ |
| | | &Tyk2 | |

### SUMMARY OF THE INVENTION

The present inventors have found that a group of compounds based either upon a 2-amino-6-carba-disubstituted pyrazine scaffold or a 2-amino-6-carba-disubstituted pyridine scaffold are JAK inhibitors.

Accordingly, in a first aspect the present invention consists in a method of inhibiting JAK in a cell, the method comprising administering to the cell an effective amount of a composition comprising a carrier and a compound of the general formula I: 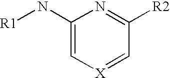
or pharmaceutically acceptable salts, hydrates, solvates, crystal forms or diastereomers thereof, wherein

X is either carbon or nitrogen

R1 is C₁₋₁₀ Alkyl, C₂₋₁₀ Alkenyl, C₂₋₁₀ Alkynyl, C₂₋₁₀ Alkylaryl, Aryl, or Heterocyclyl, or R1 with N may form a substituted or unsubstituted heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S;

R2 is selected from C₁₋₁₀ Alkyl, C₂₋₁₀ Alkenyl, C₂₋₁₀ Alkynyl, C₂₋₁₀ Alkylaryl, Aryl, Ha OH, or 6-7 membered Heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S.

In a second aspect the present invention consists in a method of inhibiting JAK in a cell, the method comprising administering to the cell an effective amount of a composition comprising a carrier and a compound of the general formula II: 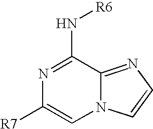
or pharmaceutically acceptable salts, hydrates, solvates, crystal forms or diastereomers thereof, wherein

R6 is C₁₋₁₀ Alkyl, C₂₋₁₀ Alkenyl, C₂₋₁₀ Alkynyl, C₂₋₁₀ Alkylaryl, Aryl, or Heterocyclyl, or R1 with N may form a substituted or unsubstituted heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S;

R7 is C₁₋₁₀ Alkyl, C₂₋₁₀ Alkenyl, C₂₋₁₀ Alkynyl, C₂₋₁₀ Alkylaryl, Aryl, Halo, OH, or Heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S.

The present invention also includes the use of compounds of formula I or II is the prophylaxis and/or treatment of JAK-associated disease states.

### DETAILED DESCRIPTION OF THE INVENTION

In a first aspect the present invention consists in a method of inhibiting JAK in a cell, the method comprising administering to the cell an effective amount of a composition comprising a carrier and a compound of the general formula I: 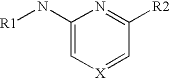
or pharmaceutically acceptable salts, hydrates, solvates, crystal forms or diastereomers thereof, wherein

X is either carbon or nitrogen

R1 is C₁₋₁₀ Alkyl, C₂₋₁₀ Alkenyl, C₂₋₁₀ Alkynyl, C₂₋₁₀ Alkylaryl, Aryl, or Heterocyclyl, or R1 with N may form a substituted or unsubstituted heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S;

R2 is selected from C₁₋₁₀ Alkyl, C₂₋₁₀ Alkenyl, C₂₋₁₀ Alkynyl, C₂-₁₀ Alkylaryl, Aryl, Halo, OH, or 6-7 membered Heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S.

In a preferred embodiment of the present invention the compound is of the general formula: 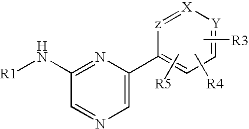
wherein one of X,Y and Z is nitrogen and the other two are carbon, or all three are carbon;

R3, R4 and R5 are the same or different and are H, halo, OH, hydroxyamide, amino, hydroxyalkyl, aminoalkylamide, alkylamide, arylamide or alkoxy.

In a more preferred embodiment when R1 with N forms a heterocycle it is preferred that the heterocycle includes two heteroatoms, preferably two nitrogen atoms.

In a more preferred embodiment of the present invention the compound is of the general formula:

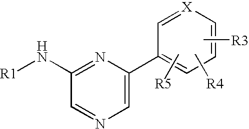

wherein X is nitrogen or carbon;

R1 is C₂-₁₀ Alkylphenyl, Phenyl, or Heterocyclyl, wherein the Alkyl, Phenyl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 member ring and in which the hetero atom is O, N or S;

R3, R4 and R5 are the same or different and are H, halo, OH, hydroxyamide, amino, hydroxyalkyl, aminoalkylamide, alkylamide, arylamide or alkoxy.

In a still further preferred embodiment the compound is selected from the compounds set out in Table 3.

In a second aspect the present invention consists in a method of inhibiting JAK in a cell, the method comprising administering to the cell an effective amount of a composition comprising a carrier and a compound of the general formula II: 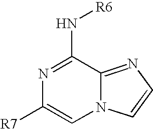
or pharmaceutically acceptable salts, hydrates, solvates, crystal forms or diastereomers thereof, wherein

R6 is C₁₋₁₀ Alkyl, C₂₋₁₀ Alkenyl, C₂₋₁₀ Alkynyl, C₂₋₁₀ Alkylaryl, Aryl, or He R1 with N may form a substituted or unsubstituted heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 member ring and in which the hetero atom is O, N or S;

R7 is C₁₋₁₀ Alkyl, C₂₋₁₀ Alkenyl, C₂₋₁₀ Alkynyl, C₂₋₁₀ Alkylaryl, Aryl, Halo, OH, or Heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 member ring and in which the hetero atom is O, N or S.

In a preferred embodiment of the present invention the compound is of the general formula:

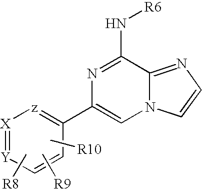

wherein one of X,Y or Z is nitrogen and the other two are carbon, or all three are carbon R8, R9 and R10 are the same or different and are H, halo, OH, hydroxyamide, amino, hydroxyalkyl, aminoalkylamide, alkylamide, arylamide or alkoxy.

In a more preferred embodiment when R1 with N forms a heterocycle it is preferred that the heterocycle includes two heteroatoms, preferably two nitrogen atoms.

In a more preferred embodiment of the present invention the compound is of the general formula: 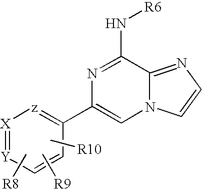
in which:

R6 is C₂₋₁₀ Alkylphenyl, Phenyl, or Heterocyclyl, wherein the Alkyl, Phenyl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S;

R3, R4 and R5 are the same or different and are H, halo, OH, hydroxyamide, amino, hydroxyalkyl, aminoalkylamide, alkylamide, arylamide or alkoxy.

In a still further preferred embodiment the compound is selected from the compounds set out in Tables 5 and 6.

In a further preferred embodiment the method is conducted in vivo. It is also preferred that the JAK is JAK1, JAK2, JAK3 or TYK2.

In a third aspect the present invention consists in a method of treating an individual suffering from a JAK-associated disease state, the method comprising administering to the individual a composition comprising a pharmaceutically acceptable carrier and a compound of the general formula: 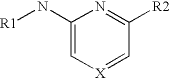
or pharmaceutically acceptable salts, hydrates, solvates, crystal forms or diastereomers thereof, wherein

X is either carbon or nitrogen

R1 is C₁₋₁₀ Alkyl, C₂₋₁₀ Alkenyl, C₂₋₁₀ Alkynyl, C₂₋₁₀ Alkylaryl, Aryl, or Heterocyclyl, or R1 with N may form a substituted or unsubstituted heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S;

R2 is selected from C₁₋₁₀ Alkyl, C₂₋₁₀ Alkenyl, C₂₋₁₀ Alkynyl, C₂₋₁₀ Alkylaryl, Aryl, Halo, OH, or 6-7 membered Heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 member ring and in which the hetero atom is O, N or S.

In a preferred embodiment of the present invention the compound is of the general formula:

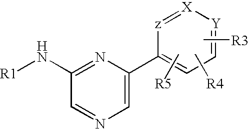

wherein one of X,Y and Z is nitrogen and the other two are carbon, or all three are carbon;

R3, R4 and R5 are the same or different and are H, halo, OH, hydroxyamide, amino, hydroxyalkyl, aminoalkylamide, alkylamide, arylamide or alkoxy.

In a more preferred embodiment when R1 with N forms a heterocycle it is preferred that the heterocycle includes two heteroatoms, preferably two nitrogen atoms.

In a more preferred embodiment of the present invention the compound is of the general formula:

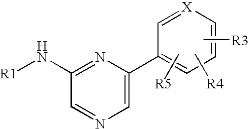

wherein X is nitrogen or carbon;

R1 is C₂₋₁₀ Alkylphenyl, Phenyl, or Heterocyclyl, wherein the Alkyl, Phenyl, and Heterocyclyl, is optionally substituted with one to three members selected from the group consisting of chloro, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S;

R3, R4 and R5 are the same or different and are H, halo, OH, hydroxyamide, amino, hydroxyalkyl, aminoalkylamide, alkylamide, arylamide or alkoxy.

In a still further preferred embodiment the compound is selected from the compounds set out in Table 3.

In a fourth aspect the present invention consists in a method of treating an individual suffering from a JAK-associated disease state, the method comprising administering to the individual a composition comprising a pharmaceutically acceptable carrier and a compound of the general formula: 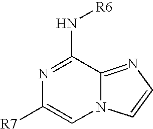
or pharmaceutically acceptable salts, hydrates, solvates, crystal forms or diastereomers thereof, wherein

R6 is C₁₋₁₀ Alkyl, C₂₋₁₀ Alkenyl, C₂₋₁₀ Alkynyl, C₂₋₁₀ Alkylaryl, Aryl, or Heterocyclyl, or R1 with N may form a substituted or unsubstituted heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 member ring and in which the hetero atom is O, N or S;

R7 is C₁₋₁₀ Alkyl, C₂₋₁₀ Alkenyl, C₂₋₁₀ Alkynyl, C₂₋₁₀ Alkylaryl, Aryl, Halo, OH, or Heterocyclyl, wherein the Alkyl, Alkenyl, Alkynyl, Alkylaryl, Aryl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S.

In a preferred embodiment of the present invention the compound is of the general formula:

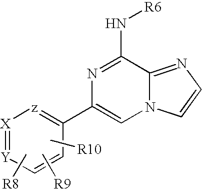

wherein one of X,Y or Z is nitrogen and the other two are carbon, or all three are carbon

R8, R9 and R10 are the same or different and are H, halo, OH, hydroxyamide, amino, hydroxyalkyl, aminoalkylamide, alkylamide, arylamide or alkoxy.

In a more preferred embodiment when R1 with N forms a heterocycle it is preferred that the heterocycle includes two heteroatoms, preferably two nitrogen atoms.

In a more preferred embodiment of the present invention the compound is of the general formula: 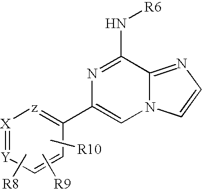
in which:

R6 is C₂₋₁₀ Alkylphenyl, Phenyl, or Heterocyclyl, wherein the Alkyl, Phenyl, and Heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl in which heterocycle is a 5-7 membered ring and in which the hetero atom is O, N or S;

R3, R4 and R5 are the same or different and are H, halo, OH, hydroxyamide, amino, hydroxyalkyl, aminoalkylamide, alkylamide, arylamide or alkoxy.

In a still further preferred embodiment the compound is selected from the compounds set out in Tables 5 and 6.

In a further preferred embodiment the disease state involves JAK1, JAK2, JAK3 or TYK2.

In a preferred embodiment of the present invention the disease state is selected from the group consisting of Atopy, such as Allergic Asthma, Atopic Dermatitis (Eczema), and Allergic Rhinitis; Cell Mediated Hypersensitivity, such as Allergic Contact Dermatitis and Hypersensitivity Pneumonitis; Rheumatic Diseases, such as Systemic Lupus Erythematosus (SLE), Rheumatoid Arthritis, Juvenile Arthritis, Sjögren's Syndrome, Scleroderma, Polymyositis, Ankylosing Spondylitis, Psoriatic Arthritis; Other autoimmune diseases such as Type I diabetes, autoimmune thyroid disorders, and Alzheimer's disease; Viral Diseases, such as Epstein Barr Virus (EBV), Hepatitis B, Hepatitis C, HIV, HTLV 1, Varicella-Zoster Virus (VZV), Human Papilloma Virus (HPV), Cancer, such as Leukemia, Lymphoma and Prostate Cancer.

In further aspects the present invention provides the use of the compounds described in the preparation of medicaments for the treatment of JAK-associated disease states.

As used herein the term “JAK”, “JAK kinase” or “JAK family” refers to protein tyrosine kinases which possess the characterizing features of JAK1, JAK2, JAK3 and TYK as described herein.

As used herein the term “JAK-associated disease state” refers to those disorders which result from aberrant JAK activity, and/or which are alleviated by inhibition of one or more of these enzymes.

The present invention provides pharmaceutical compositions comprising at least one of the compounds of the formula I or II capable of treating a JAK-associated disorder in an amount effective therefor, and a pharmaceutically acceptable vehicle or diluent. The compositions of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The compounds of the formula I or II may be administered by any suitable means, for example, orally, such as in the form of tablets, capsules, granules or powders; sublingually; buccally; parenterally, such as by subcutaneous, intravenous, intramuscular, or intracistemal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The compounds may also be administered liposomally.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases and conditions associated with inflammation and infection can be treated using the method of the present invention. In a preferred embodiment, the disease or condition is one in which the actions of eosinophils and/or lymphocytes are to be inhibited or promoted, in order to modulate the inflammatory response.

The subjects treated in the above methods, in whom which JAK inhibition is desired, are mammals, including, but not limited to, cows, sheep, goats, horses, dogs, cats, guinea pigs, rats or other bovine, ovine, equine, canine, feline, rodent or murine species, and preferably a human being, male or female.

The term “therapeutically effective amount” means the amount of the subject composition that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

Examples of other therapeutic agents include the following: cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39 (i.e., CD154), fusion proteins constructed from CD40 and gp39 (CD401g and CD8gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-α inhibitors such as tenidap, anti-TNF antibodies or soluble TNF receptor, and rapamycin (sirolimus or Rapamune) or derivatives thereof.

When other therapeutic agents are employed in combination with the compounds of the present invention they may be used for example in amounts as noted in the Physician Desk Reference (PDR) or as otherwise determined by one of ordinary skill in the art.

In the treatment or prevention of conditions which require protein tyrosine kinase inhibition an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the nature of the present invention may be more clearly understood preferred forms thereof will now be described by reference to the following non-limiting Examples.

Materials and Methods

Compound Synthesis

All compounds may be prepared in a 2-step process starting from a dihalogenated heterocycle. The dihalogenated heterocyclic starting materials 2,6-dichloropyrazine and 2,6-dibromopyridine are obtained commercially. 6,8-Dibromo-imidazo-[1,2-a]-pyrazine can be prepared following the literature route (see for example, Sablayrolles, C. et al, J. Med. Chem., 1984, 27, 206).

The first step is a nucleophilic aromatic substitution to generate a monoamino-monohalo intermediate. (Scheme 1).

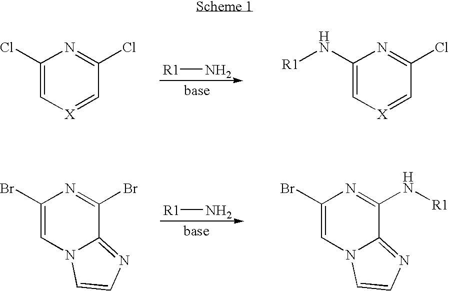

The nucleophilic aromatic substitution is typically carried out by addition of a primary amine to the di-halogenated heterocycle in a solvent such as ethanol, isopropanol, tert-butanol, dioxane, THF, DMF, toluene or xylene. The reaction is typically performed at elevated temperature in the presence of excess amine or a non-nucleophilic base such as triethylamine or diisopropylethylamine, or an inorganic base such as potassium carbonate or sodium carbonate.

The second step of the synthesis typically involves a palladium mediated cross-coupling of the monoamino-monohalo intermediate with a suitably functionalised coupling partner. Typical coupling partners are boronic acids (Suzuki coupling: see for example Miyaura, N. and Suzuki, Chem Rev. 1995, 95 2457) or stannanes (Stille coupling: see for example Stille, J. K., Angew. Chem., Int. Ed. Engl., 1986, 25, 508) (Scheme 2).

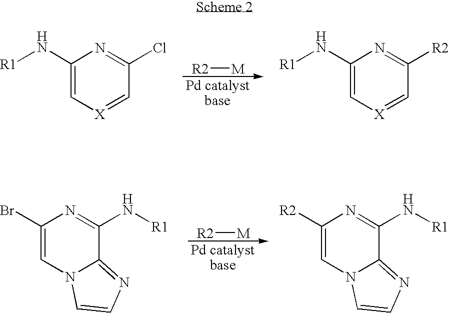

The Suzuki coupling is the preferred coupling method and is typically performed in a solvent such as DME, THF, DMF, ethanol, toluene, or 1,4-dioxane in the presence of a base such as potassium carbonate, lithium hydroxide, caesium carbonate, sodium hydroxide, potassium fluoride or potassium phosphate. The reaction may be carried out at elevated temperatures and the palladium catalyst employed may be selected from [Pd(PPh₃)₄], Pd(OAc)₂, [PdCl₂(dppf)], Pd₂(dba)₃/P(t-Bu)₃.

Representative syntheses are reported below.

### EXAMPLE 1

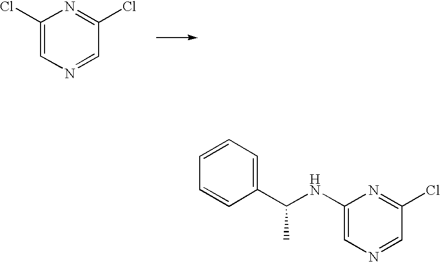

A solution of R-α-methylbenzylamine (3.64 g, 30.0 mmol) and 2,6-dichloropyrazine (1.50 g, 10.0 mmol) in dioxane (5 mL) was heated at reflux under N₂ for 48 hours. The solvent was removed and the product crystallised from toluene-hexane to give 2-(R-α-methylbenzylamino)-6-chloro-pyrazine.

¹H-n.m.r. (CDCl₃) δ1.59 (d, 3H,J=6.9 Hz, CH₃), 4.88 (q, 1H, J=6.6 Hz, CH), 5.13 (br s, 1H, NH), 7.27-7.36 (m, 5H, ArH), 7.64 (s, 1H, pyraz-H), 7.79 (s, 1H, pyraz-H).

m/z (EI) 235 (5%), 233 (16%) (M⁺)

### EXAMPLE 2

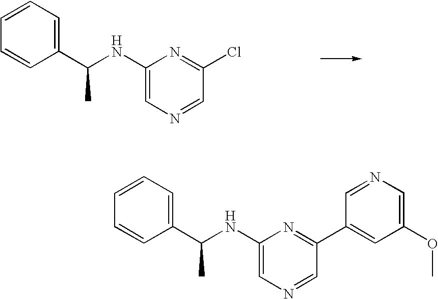

To a solution of 2-(S-α-methylbenzylamino)-6-chloro-pyrazine (120 mg, 0.51 mmol) (prepared via an analogous procedure to that outlined in Example 1), 3-methoxy-5-(4,4,5,5-tetramethyl-1,3,2-dioxaborolan-2-yl)-pyridine (144 mg, 0.61 mmol) and Pd(PPh₃)₄ (64mg, 0.05 mmol) in toluene (3 mL) was added an aqueous solution of Na₂CO₃ (0.31 mL, 2M). The resulting mixture was heated at reflux under N₂ for 16 hours. Upon cooling the mixture was diluted with water and the product extracted with EtOAc (2×15 mL). The combined organic layers were dried (Na2SO₄) and the solvent removed under reduced pressure to furnish the crude product. Column chromatography using EtOAc-hexane (5:1) as eluant furnished the purified product as a colorless oil (146mg, 93%).

¹H-n.m.r. (CDCl₃) δ1.63 (d, 3H, J=6.6 Hz, CH₃), 3.90 (3, 3H, OCH₃), 5.04 (m, 1H, CH), 5.14 (m, 1H, NH), 7.23-7.71 (m, 6H, Ar—H), 7.82 (m, 2H, pyraz-H), 8.28 (s, 1H, Ar—H), 8.33 (s, 1H, Ar—H), 8.72 (s, 1H, Ar—H).

m/z (ES) 307 (M⁺+H).

### EXAMPLE 3

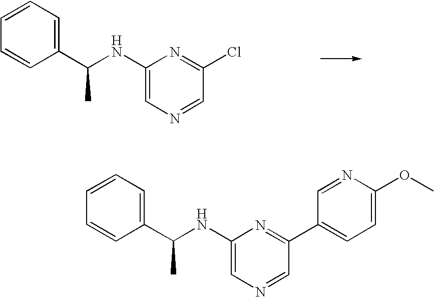

A mixture of 2-(S-α-methylbenzylamino)-6-chloro-pyrazine (100 mg, 0.43 mmol), 2-methoxypyridyl-5-boronic acid (79mg, 0.52 mmol), and Pd(PPh₃)₄ (53mg, 0.05 mmol) in toluene (3 mL) and aqueous Na₂CO₃ (0.26 mL, 2M) was treated as for Example 2 to furnish the product as a colorless oil (123 mg, 94%).

¹H-n.m.r. (CDCl₃) δ1.62 (d, 3H, J=6.3 Hz, CH₃), 3.99 (s, 3H, OCH₃), 5.01-5.06 (m, 2H, CH and NH), 6.81 (d, 1H, J=8.7 Hz, Ar—H), 7.23-7.42 (m, 5H, Ar—H), 7.72 (s, 1H, pyraz-H), 8.09 (dd, 1H, J=8.7, 2.4 Hz, Ar—H), 8.20 (s, 1H, pyraz-H), 8.73 (d, 1H, J=2.4 Hz, Ar—H).

m/z (ES) 307 (M⁺+H).

### EXAMPLE 4

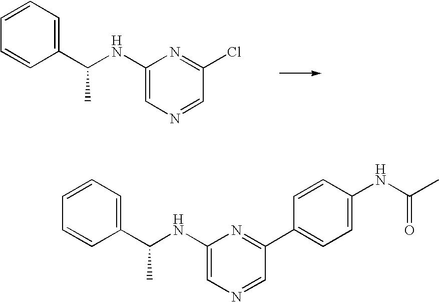

A mixture of 2-(R-α-methylbenzylamino)-6-chloro-pyrazine (66 mg, 0.28 mmol), N-[4-(4,4,5,5-tetramethyl-1,3,2-dioxaborolan-2-yl)]acetamide (88mg, 0.34 mmol) and Pd(PPh₃)₄ (16 mg, 0.02 mmol) in toluene (3 mL) and aqueous Na₂CO₃ (0.15 mL, 2M) was treated as for Example 2 to furnish the product as a colorless oil (123 mg, 94%).

1H-n.m.r. (CDCl₃) δ1.65 (d, 3H, J=6.9 Hz, CH₃), 2.21 (s, 3H, NHCH₃), 4.99 (m, 1H, CH), 6.39 (br s, 1H, NH), 7.24-7.43 (m, 5H, Ar—H), 7.65-7.78 (m, 4H, Ar—H), 7.87 (AA′XX′, 2H, Ar—H).

m/z (ES) 333 (M⁺+H).

### EXAMPLE 5

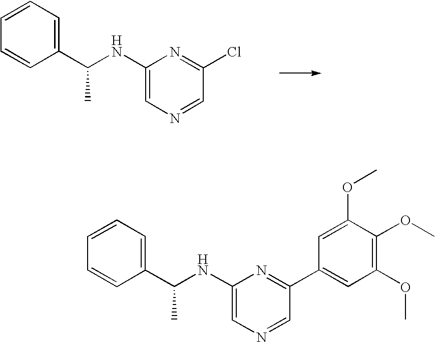

A mixture of 2-(R-α-methylbenzylamino)-6-chloro-pyrazine (500 mg, 2.2 mmol), 3,4,5-trimethoxybenzeneboronic acid (547 mg, 2.6 mmol) and Pd(PPh₃)₄ (124 mg, 0.11 mmol) in toluene (10 mL) and aqueous Na₂CO₃ (1.3 mL, 2M) was treated as for Example 2 to furnish the product as a colorless oil (574 mg, 73%). The product was obtained as pale yellow crystals by recrystallisation from methanol (m.p. 132-133°).

¹H-n.m.r. (CDCl₃) δ1.61 (d, 3H, J=6.2 Hz, CH₃), 3.88 (s, 3H, OCH₃), 3.90 (s, 6H, OCH₃), 5.02 (m, 1H, CH), 5.09 (d, 1H, J=5.9 Hz, NH), 7.10 (s, 2H, Ar—H), 7.24-7.42 (m, 5H, Ar—H), 7.74 (s, 1H, Ar—H), 8.22 (s, 1H, Ar—H).

m/z (EI) 366 (M⁺).

### EXAMPLE 6

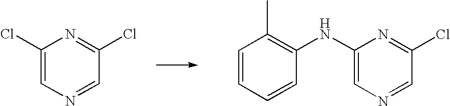

A solution of ortho-toluidine (320 mg, 3.0 mmol), 2,6-dichloropyrazine (150 mg, 1.0 mmol), bis(tributylphosphine)palladium (26 mg, 0.05mmol) and sodium tert-butoxide (144 mg, 1.5 mmol) in toluene (2 mL) was heated at 80° C. overnight. Upon cooling to room temperature, the solution was

¹H-n.m.r. (CDCl₃) δ2.29 (s, 3H, CH₃), 6.35 (br s, 1H, NH), 7.03-7.06 (m, 2H, Ar—H), 7.15-7.31 (m, 1H, Ar—H), 7.43 (d, 1H, J=7.8 Hz, Ar—H), 7.88 (s, 1H, Ar—H), 7.96 (s, 1H, Ar—H).

### EXAMPLE 7

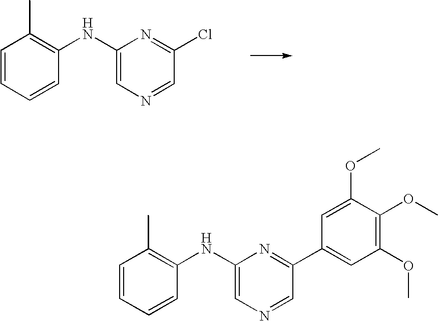

A mixture of 2-(2-methylphenyl)-6-chloro-pyrazine (1 10 mg, 0.5 mmol), 3,4,5-trimethoxybenzeneboronic acid (127 mg, 0.6 mmol) and Pd(PPh₃)₄ (62 mg, 0.05 mmol) in toluene (3 mL) and aqueous Na₂CO₃ (0.3 mL, 2M) was treated as for Example 2 to furnish the product as a white solid (147 mg, 84%).

¹H-n.m.r. (CDCl₃) δ2.27 (s, 3H, CH₃), 3.84 (s, 3H, OCH₃), 3.89 (s, 6H, OCH₃), 6.40 (br s, 1H, NH), 7.07 (d, 1H, J=7.6 Hz, Ar—H), 7.15-7.32 (m, 4H, Ar—H), 7.55 (d, 1H, J=7.8 Hz, Ar—H), 7.93 (br s, 1H, Ar—H), 8.33 (br s, 1H, Ar—H).

m/z (ES) 352 (M⁺+H).

Compound Dilution

For screening purposes, compounds were diluted in 96 well plates at a concentration of 50 μM or 20 μM. Plates were warmed at 37° C. for 30 minutes before assay.

JAK Tyrosine Kinase Domain Production

JAK kinase domains were produced in the following manner:

JAK1

The kinase domain of humanJAK1 was amplified from U937mRNA using the polymerase chain reaction with the following primers:

| | | |
|---|---|---|
| XHOI-J1 | 5′-CCG CTC GAG ACT GAA GTG | (SEQ ID NO: 1) |
| | GAC CCC ACA CAT-3′ | |
| J1-KPNI | 5′-CGG GGT ACC TTA TTT TAA | (SEQ ID NO: 2) |
| | AAG TGC TTC AAA-3′ | |

JAK1 PCR products were cloned into the pFastBac HTb expression vector (Gibco) via the Xho I and Kpn I sites. The JAK1 plasmid was then transformed into competent DH10Bac cells (Gibco), and the recombinant baculovirus produced prepared for transfection into Sf9 insect cells.

JAK2

The kinase domain of humanJAK2 was amplified from U937mRNA using the polymerase chain reaction with the following primers:

| | | |
|---|---|---|
| SALI-jk2 | 5′-ACG CGT CGA CGG TGC CTT | (SEQ ID NO: 3) |
| | TGA AGA CCG GGA T-3′ | |
| jk2-NOTI | 5′-ATA GTT TAG CGG CCG CTC | (SEQ ID NO: 4) |
| | AGA ATG AAG GTC ATT T-3′ | |

JAK2 PCR products were cloned into the pFastBac HTc expression vector (Gibco) via the Sal I and Not I sites. The JAK2 plasmid was then transformed into competent DH10Bac cells (Gibco), and the recombinant baculovirus produced prepared for transfection into Sf9 insect cells.

JAK3

The kinase domain of humanJAK3 was amplified from U937mRNA using the polymerase chain reaction with the following primers:

| | | |
|---|---|---|
| XHOI-J3 | 5′-CCG CTC GAG TAT GCC TGC | (SEQ ID NO: 5) |
| | CAA GAC CCC ACG-3′ | |
| J3-KPNI | 5′-CGG GGT ACC CTA TGA AAA | (SEQ ID NO: 6) |
| | GGA CAG GGA GTG-3′ | |

JAK3 PCR products were cloned into the pFastBac HTb expression vector (Gibco) via the Xho I and Kpn I sites. The JAK3 plasmid was then transformed into competent DH10Bac cells (Gibco), and the recombinant baculovirus produced prepared for transfection into Sf9 insect cells.

TYK2

The kinase domain of humanTYK2 was amplified from A549 mRNA using the polymerase chain reaction with the following primers:

| | | |
|---|---|---|
| HT2EK | 5′-GGA GCA CTC GAG ATG GTA | (SEQ ID NO: 7) |
| | GCA CAC AAC CAG GTG-3′ | |
| ITY2.2R | 5′-GGA GCA GGA ATT CCG GCG | (SEQ ID NO: 8) |
| | CTG CCG GTC AAA TCT GG-3′ | |

TYK2 PCR products were cloned into pBlueBacHis2A (Invitrogen) via the EcoRI site. The recombinant TYK2 baculovirus produced was prepared for transfected into Sf9 insect cells.

Large Scale Production of Kinase Domains

Baculovirus preparations from each of the JAK family members were infected into five litres of High Five cells (Invitrogen) grown in High Five serum free medium (Invitrogen) to a cell density of approximately 1-2×10⁶ cells/ml. Cells are infected with virus at a MOI of 0.8-3.0. Cells were harvested and lysed. JAK kinase domains were purified by affinity chromatography on a Probond (Invitrogen) nickel chelate affinity column.

Assay Protocols

Kinase assays were performed in a 96 well capture-based ELISA assay, using approximately 1.5 μg of affinity purified PTK domain in the presence of 50 mM HEPES, pH 7.5, 10 mM MgCl₂, 150 mM NaCl and 10-20 μM ATP. The biotinylated substrate biotin-EGPWLEEEEEAYGWMDF-NH₂ (SEQ ID NO:9) (final concentration 5 μM) was used as substrate, and tyrosine phosphorylation was quantitated following transfer to an avidin coated ELISA plate using peroxidase-linked anti-phospho-tyrosine antibody PY20.

Inhibitors were added to the assays fifteen minutes prior to the addition of ATP. Inhibitors were added in aqueous DMSO, with DMSO concentrations never exceeding 1%.

Cellular assays were performed as follows:

Cell suspensions were prepared by harvesting cells from culture. Cell used in this test should be in later log phase growth and high viability. Cells were diluted in correct growth medium to 1.1× final concentration (from 50000 cell/ml to 200,000 cell/ml, depending on cell line). 90 μL was added to samples, diluted in PBS to 10× final concentration in flat-bottom 96-well plates (10 μL). After incubation for 40 hr in 37° C. 5% CO₂ incubator, MTT 5 mg/ml (in PBS, filter sterile) 20 μl per well was added. The plates were returned to incubator for another 6 hours. Lysis Buffer (10% SDS, 0.01N HCl) 100 μl per well was added and the plate put back in incubator overnight. The plate was then read at 590 nm.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Tables

Table 3: Selected 2-amino-6-carba-disubstituted pyrazines and 2-amino-6-carba-disubstituted pyridines possessing JAK inhibitory activity

Compounds that exhibited a capacity to inhibit 50% of JAK activity at a concentration of 50 μM (measured under standard conditions, see Methods), are shown.

Table 4: Selected 2-amino-6-carba-disubstituted pyrazines possessing inhibitory activity on certain cells

Compounds that exhibited a capacity to inhibit 50% of cellular growth (measured under standard conditions, see Methods), are shown.

Table 5 and 6: 6-carba-8-amino-disubstituted imidazo-[1,2-a]-pyrazine possessing JAK inhibitory activity

Compounds that exhibited a capacity to inhibit 50% of JAK activity at a concentration of 50 μM (measured under standard conditions, see Methods), are shown.

### REFERENCES

Spiotto M T, and Chung T D. (2000) STAT3 mediates IL-6-induced growth inhibition in the human prostate cancer cell line LNCaP. Prostate 42 88-98

Sadowski H B, Stone J C, Pawson T. (1986) A noncatalytic domain conserved among cytoplasmic protein-tyrosine kinases modifies the kinase function and transforming activity of Fujinami sarcoma virus p130^{gag-fps}. Mol Cell Biol 6 4396-408.

Harpur A G, Andres A C, Ziemiecki A, Aston R. R. and Wilks, A. F.,. (1992) JAK2, a third member of the JAK family of protein tyrosine kinases. Oncogene; 7 1347-53.

Kozma S C, Redmond S M S, Xiao-Chang F, et al. (1988) Activation of the receptor kinase domain of the trk oncogene by recombination with two different cellular sequences. EMBO 7 147-54.

Wilks A F, Harpur A G, Kurban R R, Ralph S J, Zurcher G, Ziemiecki A. (1991) Two novel protein-tyrosine kinases, each with a second phosphotransferase-related catalytic domain, define a new class of protein kinase. Mol Cell Biol. 11 2057-65.

Wilks A F, Kurban R R. (1988) Isolation and structural analysis of murine c-fes cDNA clones. Oncogene 3 289-94.

TABLE 3

| CHEMISTRY | CYT ID # | JAK1 | JAK2 | JAK3 | TYK2 | AbI | Hck |
|---|---|---|---|---|---|---|---|
| 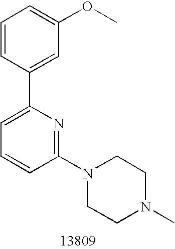 | CYT13809 | + | + | + | + | − | − |
| 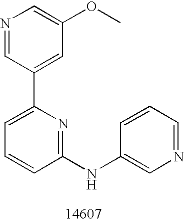 | CYT14607 | + | + | + | + | − | − |
| 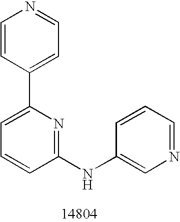 | CYT14804 | + | + | + | + | − | − |
| 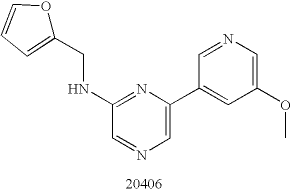 | CYT20406 | + | + | + | + | − | − |
| 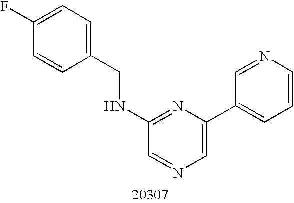 | CYT20307 | + | + | + | + | − | − |
| 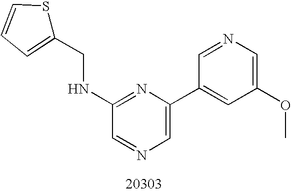 | CYT20303 | + | + | + | + | − | − |
| 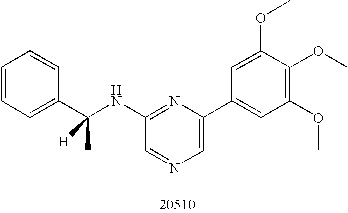 | CYT20510 | + | + | + | + | − | − |
| 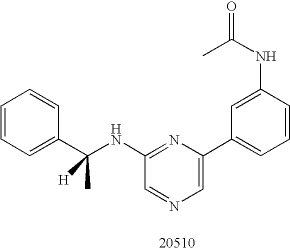 | CYT20508 | + | + | + | + | − | − |
| 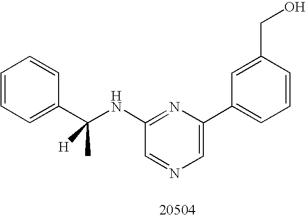 | CYT20504 | + | + | + | + | − | − |
| 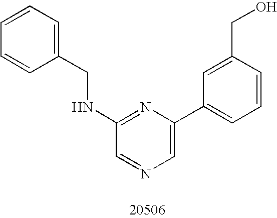 | CYT20506 | + | + | + | + | − | − |
| 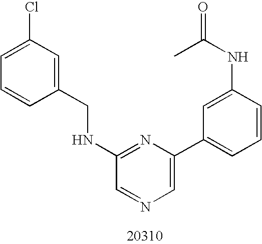 | CYT20310 | + | + | + | + | − | − |
| 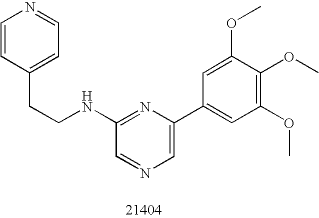 | CYT21404 | + | + | + | + | − | − |
| 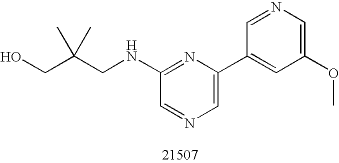 | CYT21103 | + | + | + | + | − | − |
| 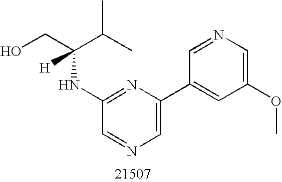 | CYT21507 | + | + | + | + | − | − |
| 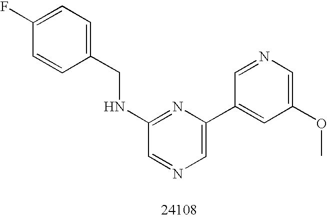 | CYT24108 | + | + | + | + | − | − |
| 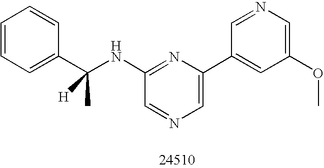 | CYT24510 | + | + | + | + | − | − |
| 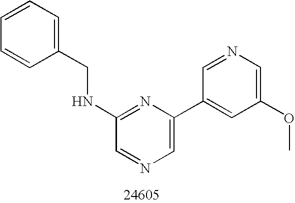 | CYT24605 | + | + | + | +− | − | |
| 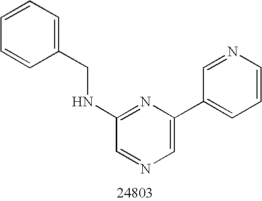 | CYT24803 | + | + | + | +− | − | |
| 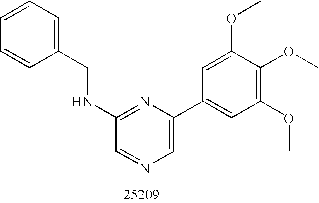 | CYT25209 | + | + | + | + | − | − |
| 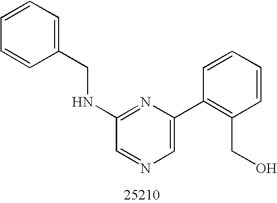 | CYT25210 | + | + | + | + | − | − |
| 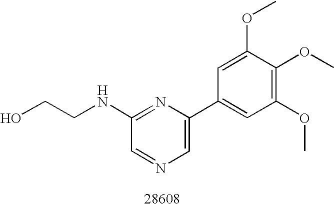 | CYT28608 | + | + | + | + | − | − |
| 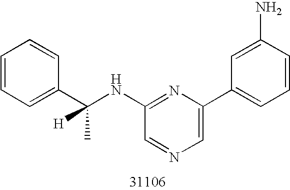 | CYT31106 | + | + | + | + | − | − |
| 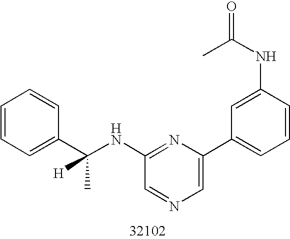 | CYT32102 | + | + | + | + | − | − |
| 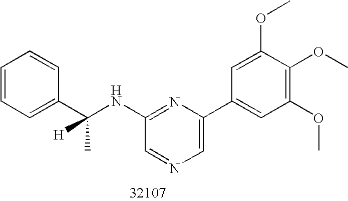 | CYT32107 | + | + | + | + | − | − |
| 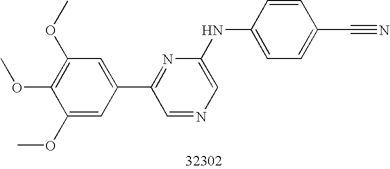 | CYT32302 | + | + | + | + | − | − |
| 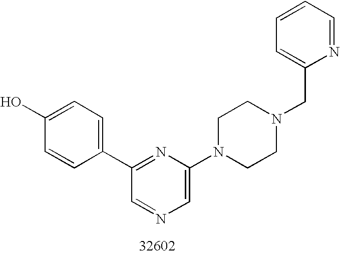 | CYT32602 | + | + | + | + | − | − |
| 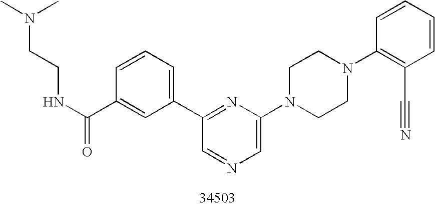 | CYT34503 | + | + | + | + | − | − |
| 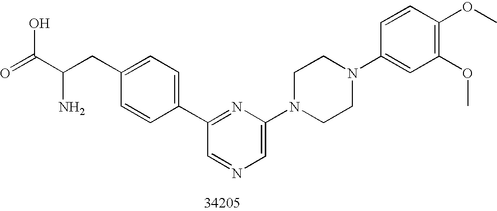 | CYT34205 | + | + | + | + | − | − |
| 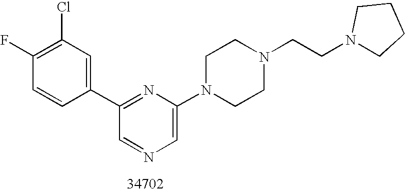 | CY734702 | + | + | + | + | − | − |

TABLE 4

| | T-cell activation | Jurkat IC50 | TSU IC50 |
|---|---|---|---|
| CHEMISTRY | IC50 (uM) | (uM) | (uM) |
| 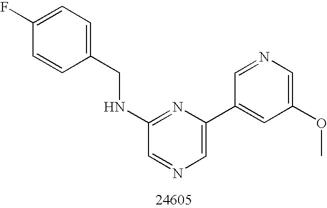 | 30 | 50 | 50 |
| 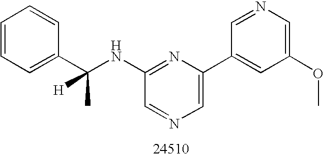 | 15 | 50 | 20 |
| 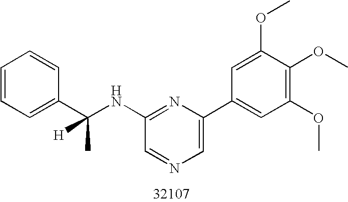 | 10 | 20 | 10 |
| 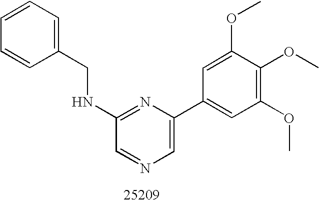 | 15 | 20 | 10 |
| 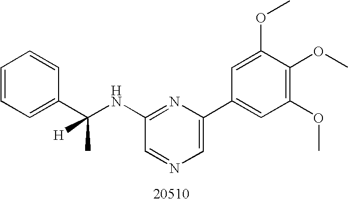 | 12 | 35 | 10 |
| 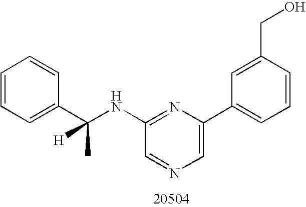 | 20 | 20 | 15 |
| 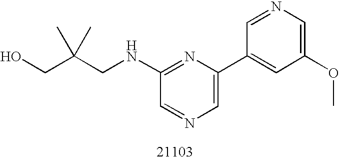 | 15 | 40 | 40 |
| 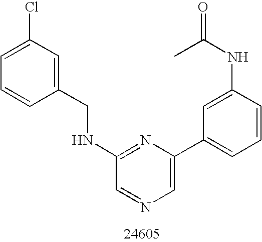 | 30 | 40 | 5 |

TABLE 5

| CHEMISTRY | JAK1 | JAK2 | JAK3 | TYK2 | AbI |
|---|---|---|---|---|---|
| 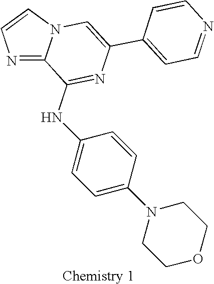 | + | + | + | + | − |
| 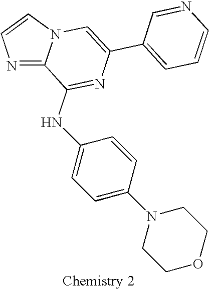 | + | + | + | + | − |
| 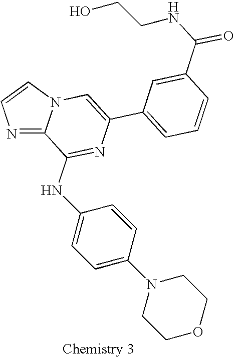 | + | + | + | + | − |
| 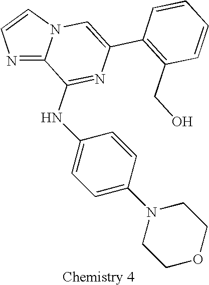 | + | + | + | + | − |
| 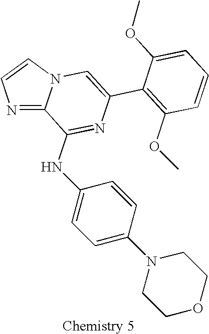 | + | + | + | + | − |
| 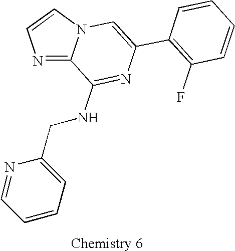 | + | + | + | + | − |
| 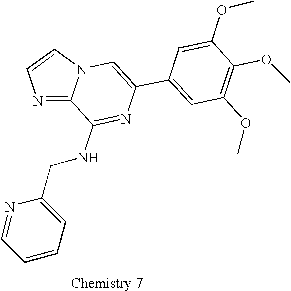 | + | + | + | + | − |
| 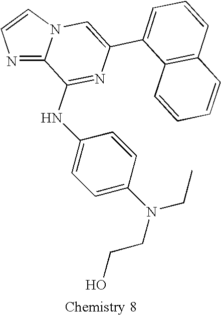 | + | + | + | + | − |
| 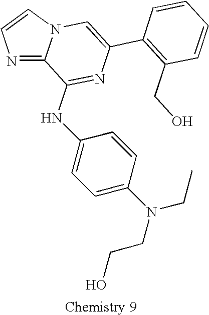 | + | + | + | + | − |
| 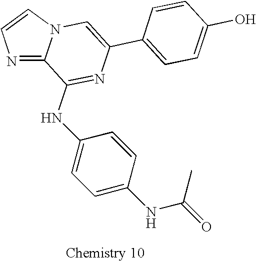 | + | + | + | + | − |
| 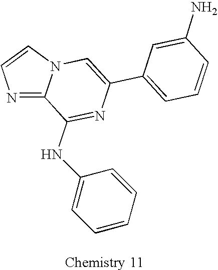 | + | + | + | + | − |
| 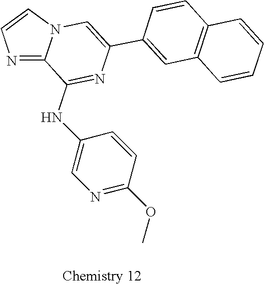 | + | + | + | + | − |
| 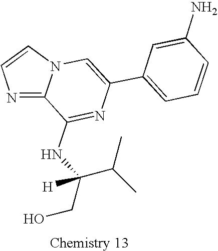 | + | + | + | + | − |
| 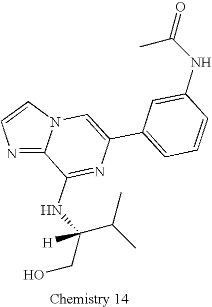 | + | + | + | + | − |
| 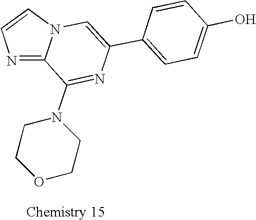 | + | + | + | + | − |
| 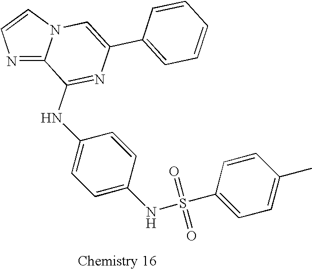 | + | + | + | + | − |
| 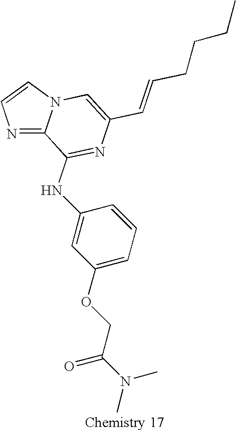 | + | + | + | + | − |
| 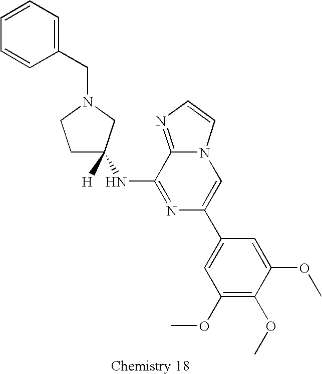 | + | + | + | + | − |
| 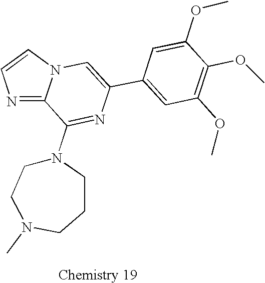 | + | + | + | + | − |
| 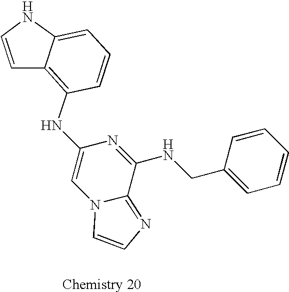 | + | + | + | + | − |
| 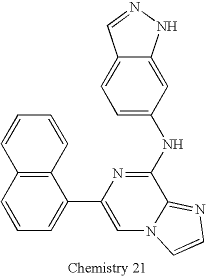 | + | + | + | + | − |
| 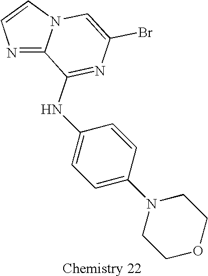 | + | + | + | + | − |
| 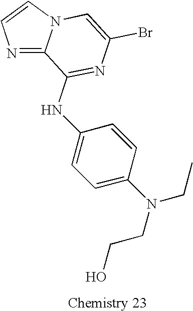 | + | + | + | + | − |
| 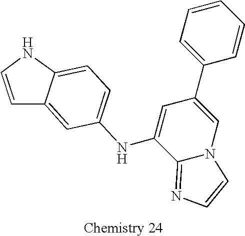 | + | + | + | + | − |
| 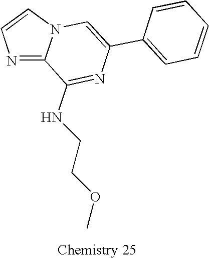 | + | + | + | + | − |

TABLE 6

| CHEMISTRY | Jak1 | Jak2 | Jak3 | Tyk2 |
|---|---|---|---|---|
| 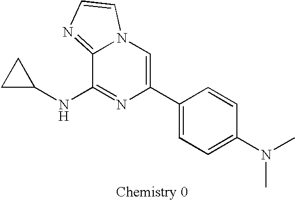 | − | − | + | − |
| 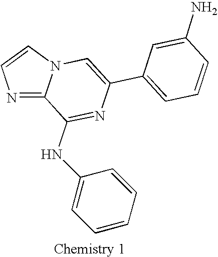 | + | + | − | + |
| 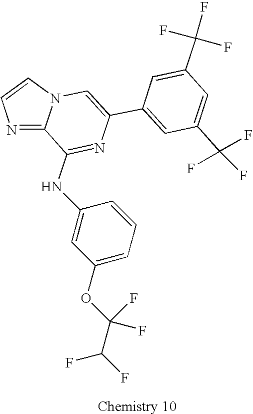 | + | − | − | − |
| 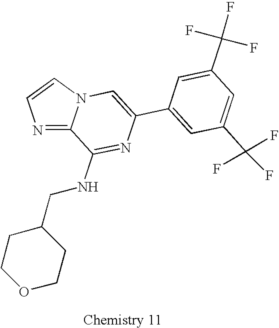 | − | + | − | − |
| 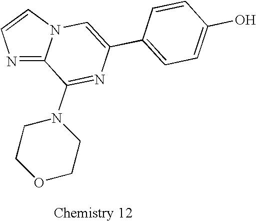 | − | + | − | − |
| 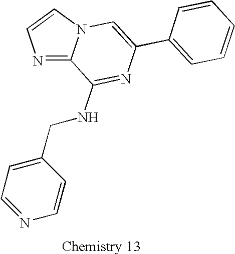 | + | − | − | − |
| 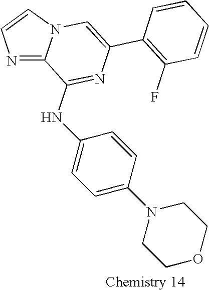 | + | + | − | + |
| 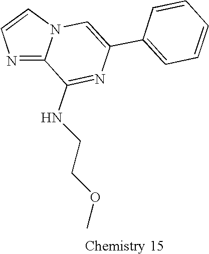 | + | + | − | − |
| 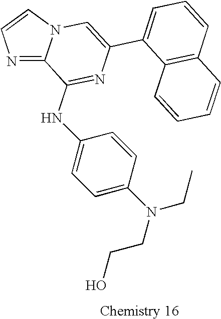 | + | + | − | − |
| 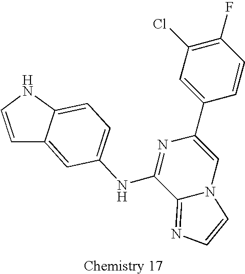 | + | − | − | + |
| 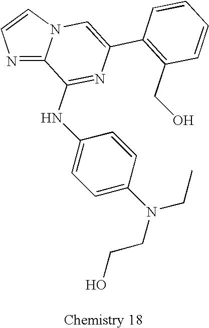 | + | + | − | + |
| 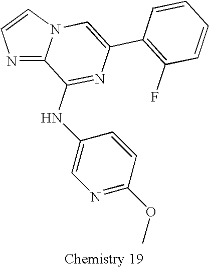 | + | + | − | − |
| 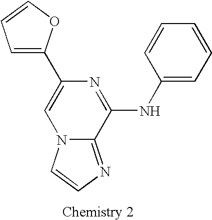 | − | + | − | + |
| 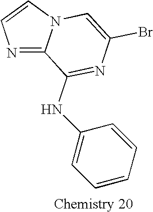 | + | + | − | + |
| 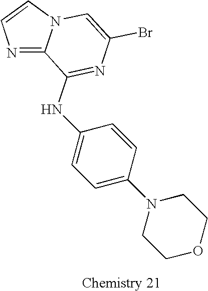 | + | + | − | + |
| 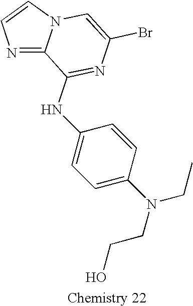 | + | + | − | + |
| 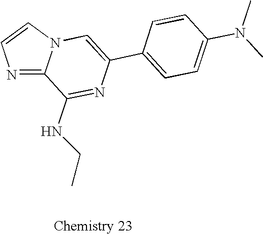 | + | − | − | − |
| 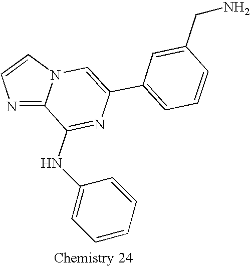 | + | + | − | + |
| 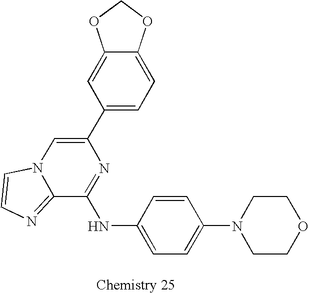 | + | + | − | + |
| 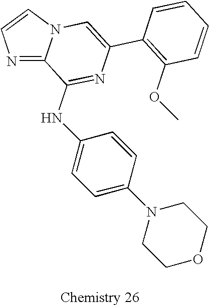 | + | + | − | + |
| 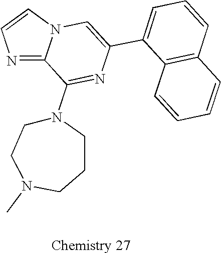 | + | − | − | − |
| 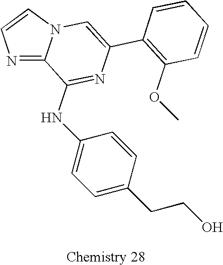 | + | + | − | + |
| 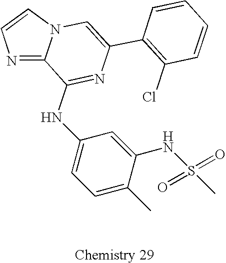 | + | + | − | + |
| 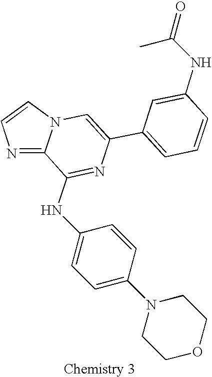 | + | + | − | + |
| 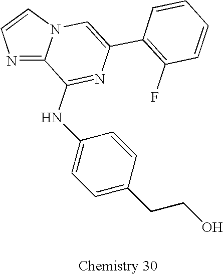 | + | + | + | + |
| 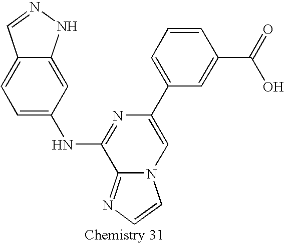 | + | + | − | + |
| 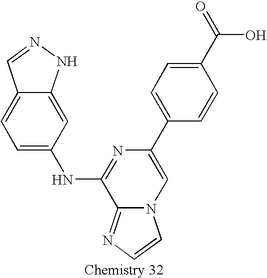 | + | + | − | + |
| 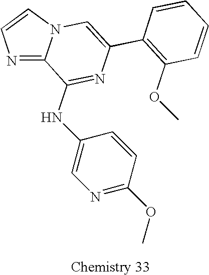 | + | + | − | + |
| 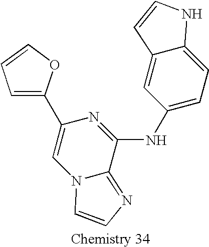 | + | + | − | + |
| 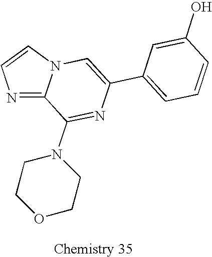 | − | + | − | − |
| 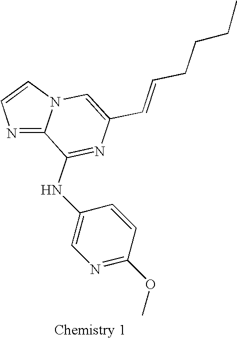 | − | + | − | − |
| 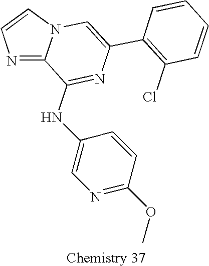 | − | + | − | − |
| 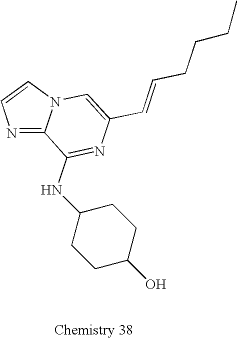 | − | − | + | − |
| 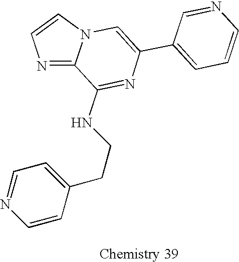 | − | − | + | − |
| 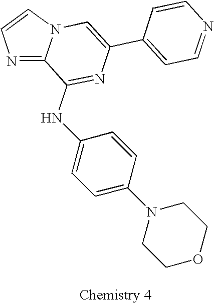 | + | + | − | + |
| 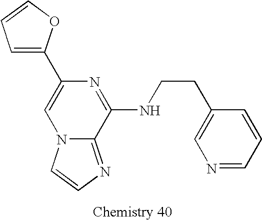 | − | − | + | − |
| 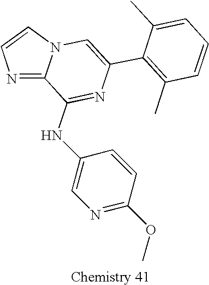 | − | − | + | − |
| 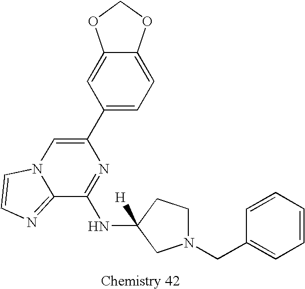 | − | − | − | + |
| 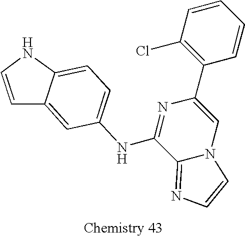 | + | + | − | + |
| 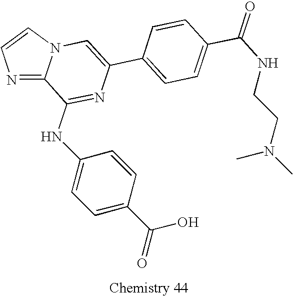 | + | + | + | + |
| 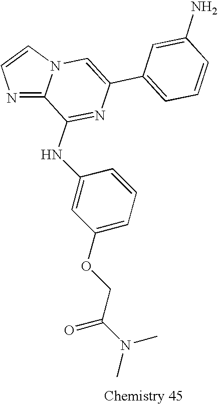 | + | + | + | + |
| 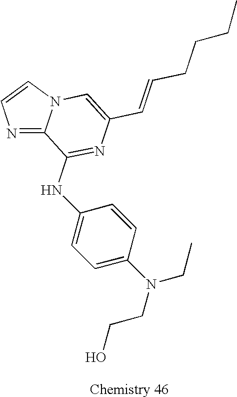 | + | + | + | + |
| 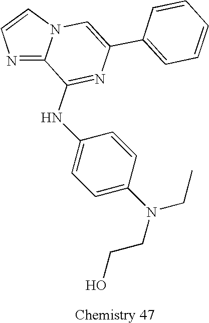 | + | + | + | + |
| 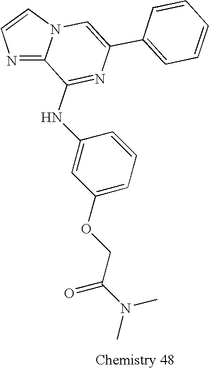 | + | + | − | + |
| 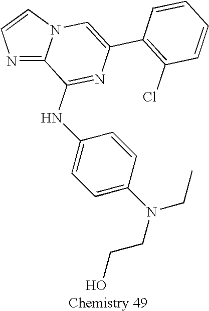 | + | + | + | + |
| 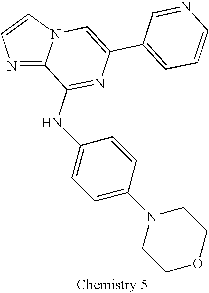 | + | + | − | + |
| 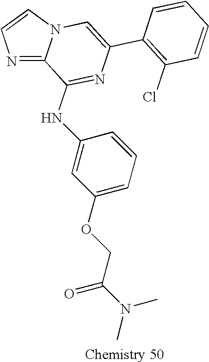 | + | + | + | + |
| 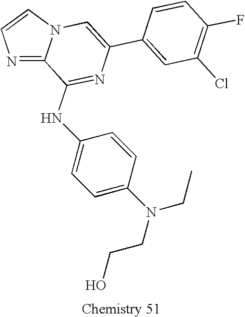 | + | + | − | + |
| 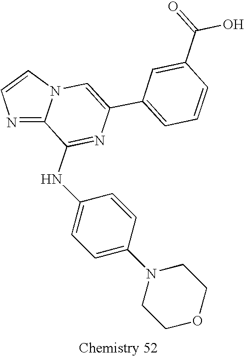 | + | + | + | + |
| 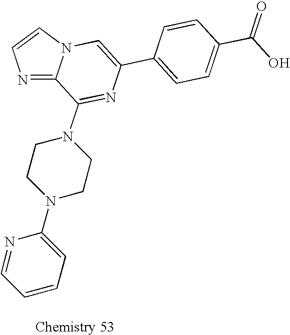 | + | − | − | − |
| 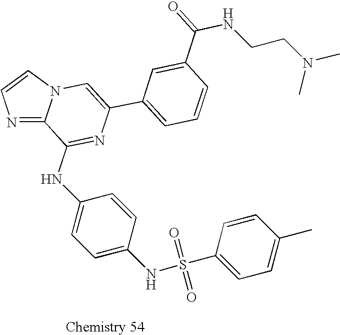 | + | + | − | − |
| 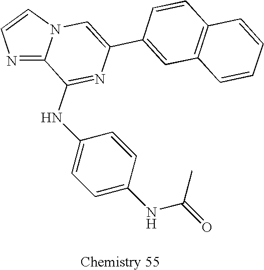 | + | + | − | + |
| 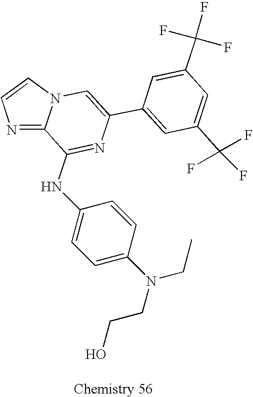 | − | + | − | − |
| 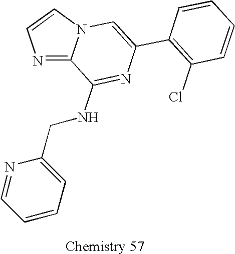 | − | + | − | − |
| 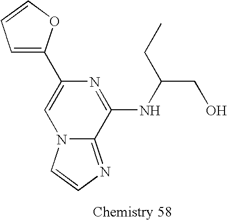 | + | + | + | − |
| 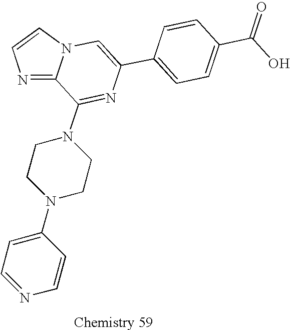 | − | + | − | − |
| 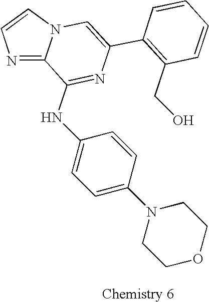 | + | + | − | − |
| 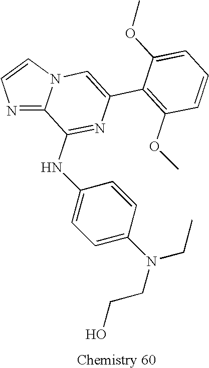 | + | + | − | − |
| 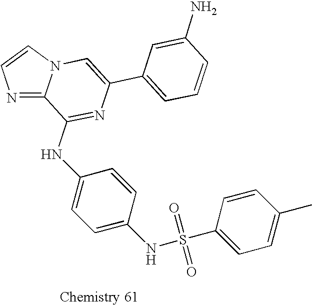 | + | + | − | + |
| 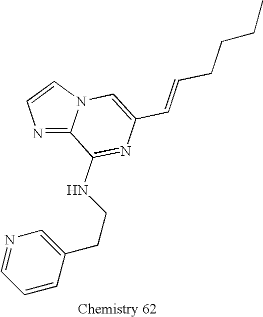 | − | − | + | − |
| 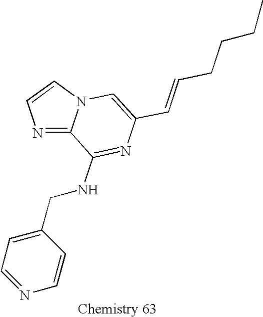 | − | − | + | − |
| 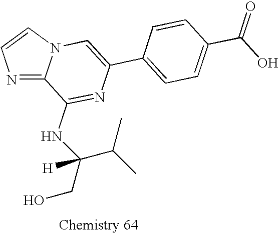 | − | + | + | − |
| 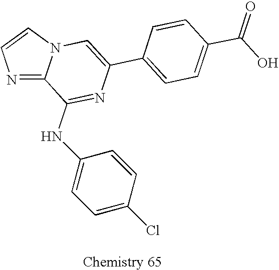 | + | + | + | + |
| 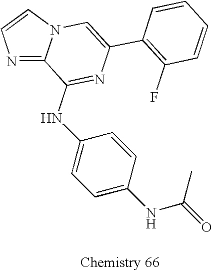 | + | + | + | + |
| 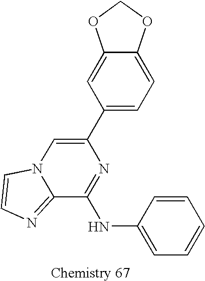 | + | + | − | + |
| 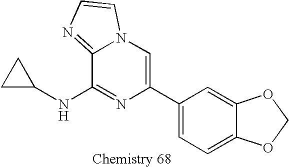 | + | − | − | + |
| 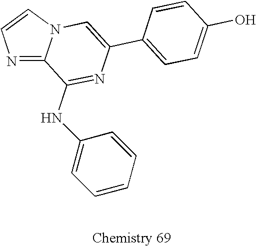 | + | + | − | − |
| 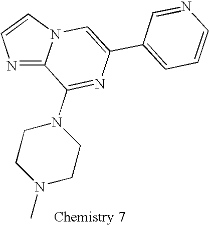 | + | − | − | − |
| 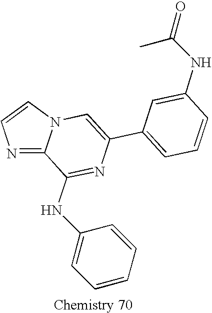 | + | + | + | + |
| 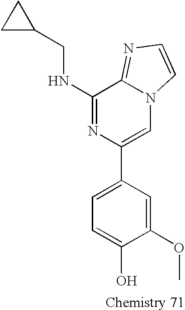 | + | − | − | − |
| 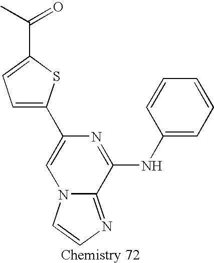 | + | + | + | + |
| 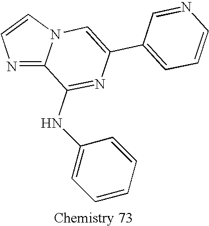 | + | + | + | + |
| 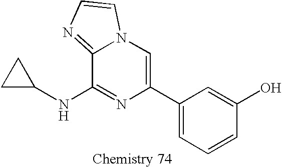 | + | + | − | + |
| 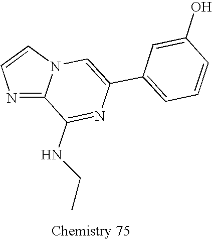 | + | + | + | − |
| 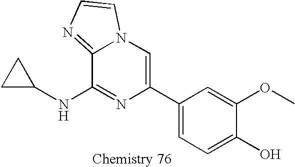 | + | + | + | − |
| 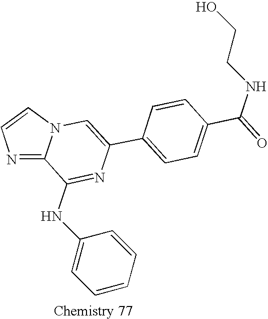 | + | + | − | + |
| 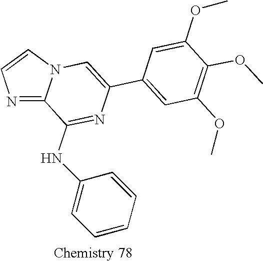 | + | + | − | − |
| 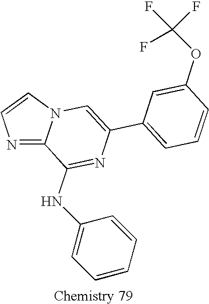 | − | + | − | − |
| 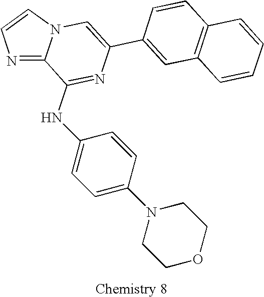 | + | + | − | − |
| 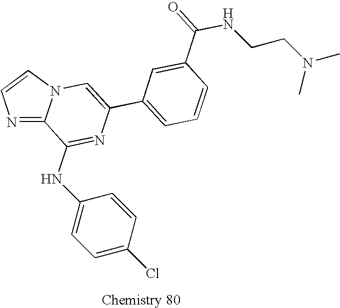 | + | + | − | − |
| 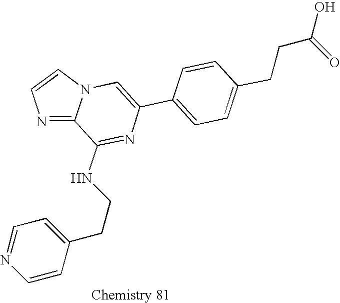 | − | − | + | − |
| 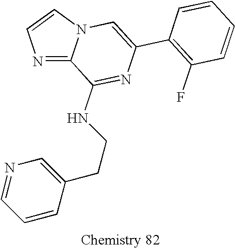 | + | + | + | − |
| 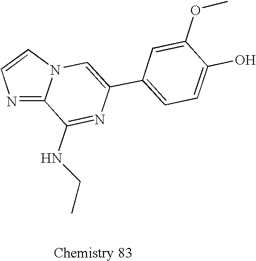 | + | + | + | − |
| 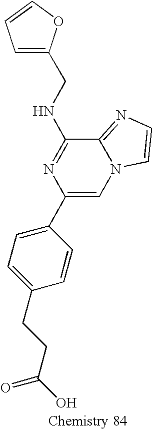 | − | − | + | + |
| 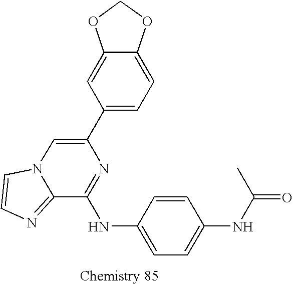 | + | + | + | + |
| 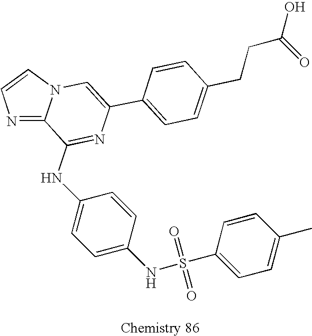 | + | + | − | + |
| 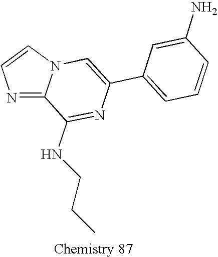 | + | − | − | − |
| 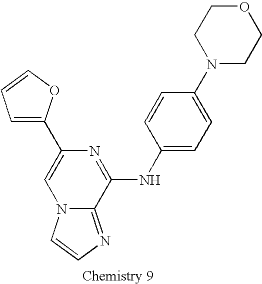 | + | + | − | + |

1-34. (canceled)

35. A method of inhibiting JAK in a cell, which comprises administering to the cell an effective amount of a composition comprising a carrier and a compound of the general formula II: 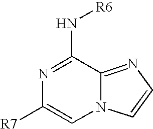
or pharmaceutically acceptable salts, hydrates, solvates, crystal forms or diastereomers thereof, wherein
R6 is C₁₋₁₀ alkyl, C₂₋₁₀ alkenyl, C₂₋₁₀ alkynyl, C₂₋₁₀ alkylaryl, aryl, or heterocycle, or R6 with N may form a substituted or unsubstituted heterocycle, wherein the alkyl, alkenyl, alkynyl, alkylaryl, aryl, and heterocycle, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl wherein heterocycle is a 5-7 membered ring and wherein the hetero atom is O, N or S;
R7 is C₁₋₁₀ alkyl, C₂₋₁₀ alkenyl, C₂₋₁₀ alkynyl, C₂₋₁₀ alkylaryl, aryl, halo, OH, or heterocycle, wherein the alkyl, alkenyl, alkynyl, alkylaryl, aryl, and heterocycle, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl wherein heterocycle is a 5-7 member ring and wherein the hetero atom is O, N or S.

36. A method as claimed in claim 35 wherein the compound is of the general formula: 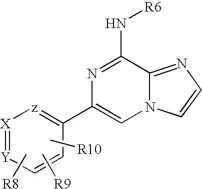
wherein one of X, Y or Z is nitrogen and the other two are carbon, or all three are carbon
R8, R9 and R10 are the same or different and are H, halo, OH, hydroxyamide, amino, hydroxyalkyl, aminoalkylamide, alkylamide, arylamide or alkoxy.

37. A method as claimed in claim 36 wherein R6 with N forms a heterocycle.

38. A method as claimed in claim 37 wherein the heterocycle includes two heteroatoms, preferably two nitrogen atoms.

39. A method as claimed in claim 35 wherein the compound is, of the general formula: 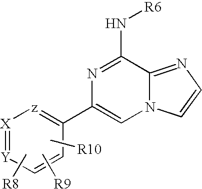
wherein:
R6 is C₂₋₁₀ alkylphenyl, phenyl, or heterocycle, wherein the alkyl, phenyl, and heterocycle, is optionally substituted with one to three members selected from the group consisting of chloro, amino, hydroxy, hydroxyalkyl, alkylamide, arylamide, hydroxyalkylamide, nitrilo, aminoalkylamide, nitriloaryl, alkoxy (in particular methoxy), heterocyclic alkyl wherein heterocycle is a 5-7 member ring and wherein the hetero atom is O, N or S;
R3, R4 and R5 are the same or different and are H, halo, OH, hydroxyamide, amino, hydroxyalkyl, aminoalkylamide, alkylamide, arylamide or alkoxy.

40. A method as claimed in claim 35 wherein the compound is selected from the group consisting of:

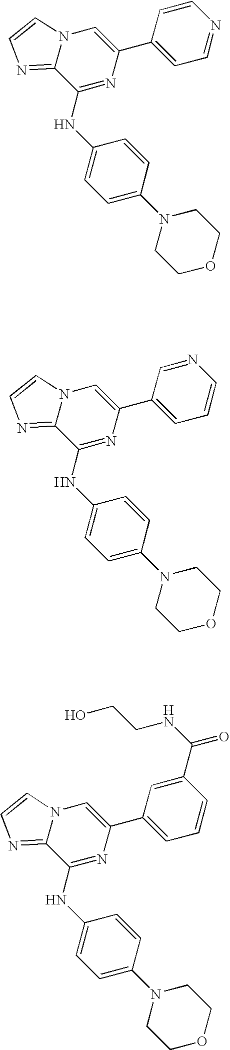

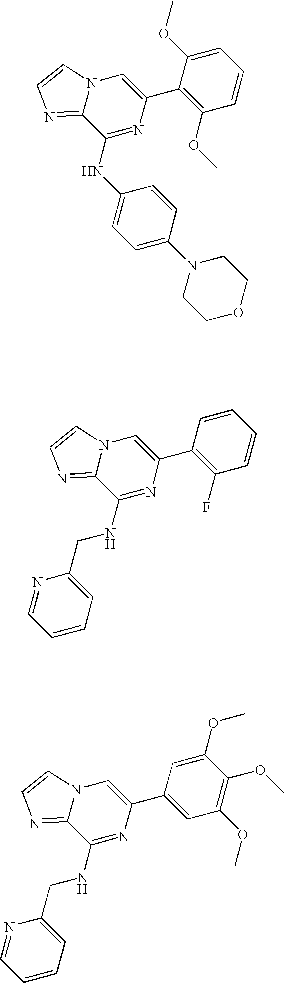

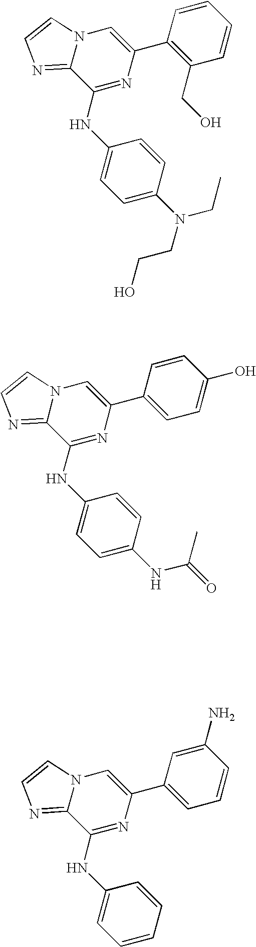

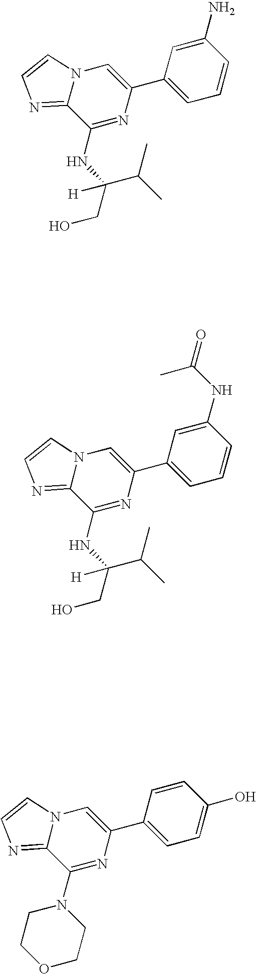

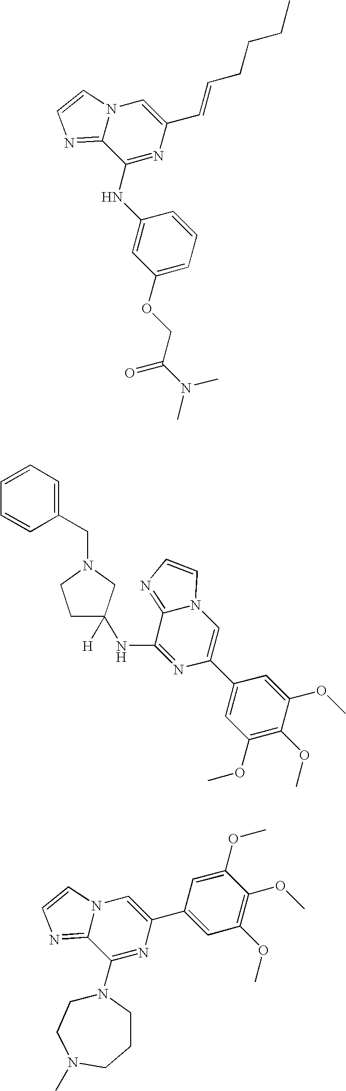

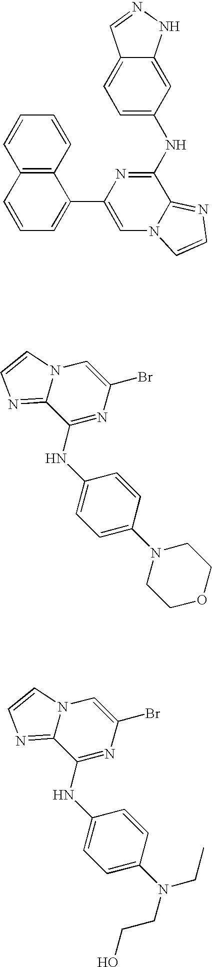

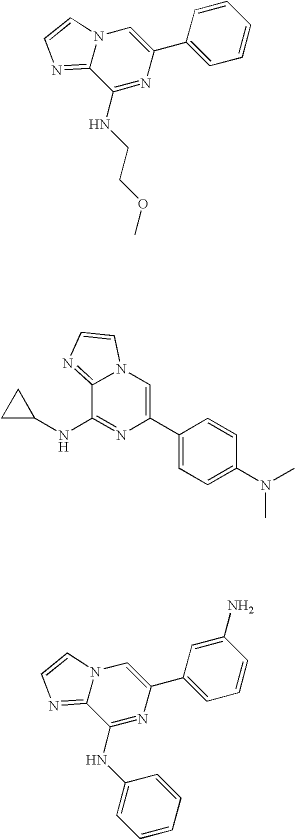

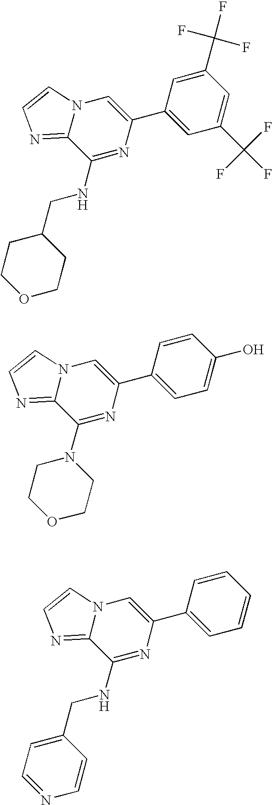

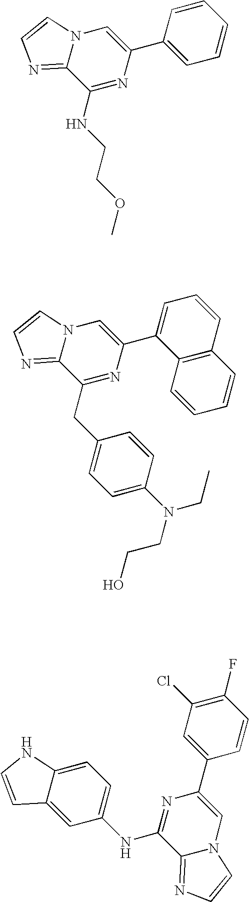

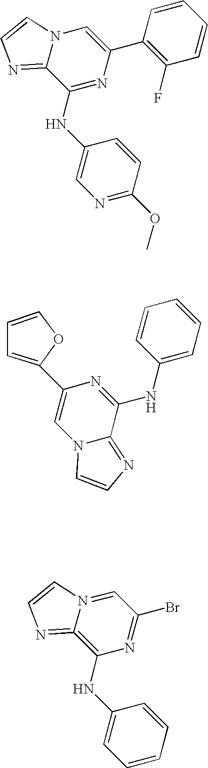

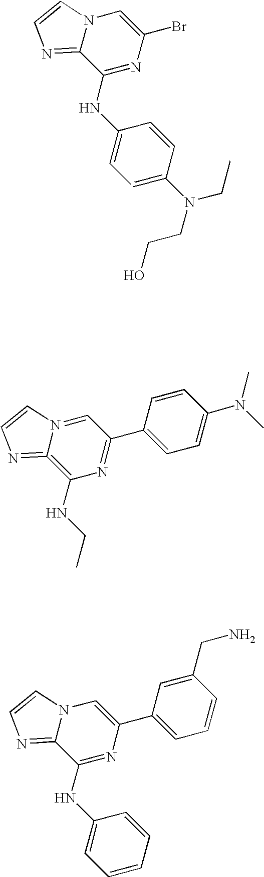

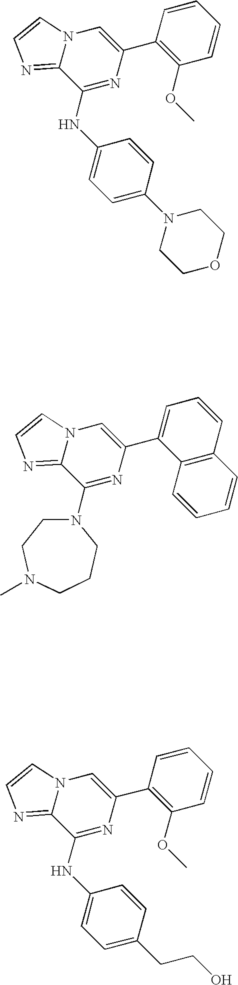

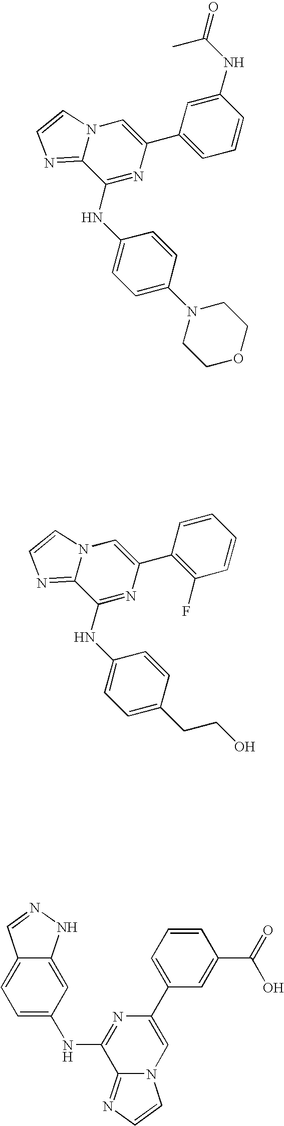

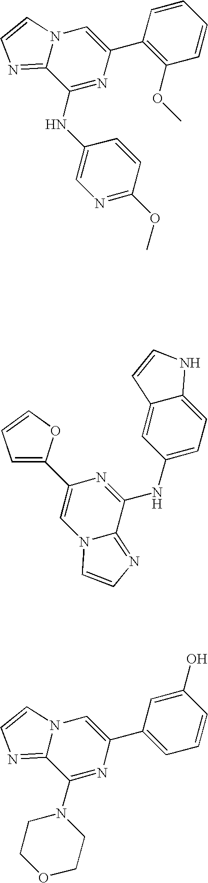

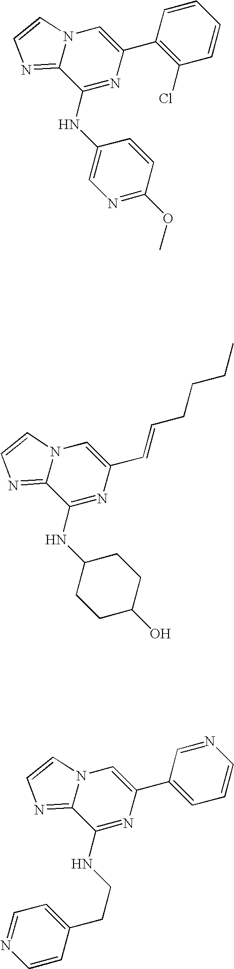

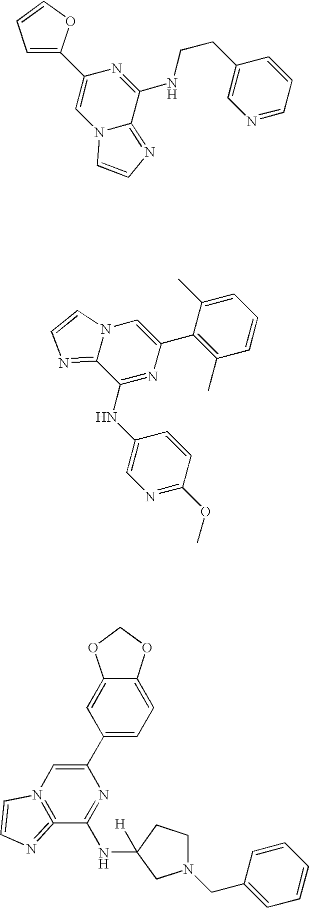

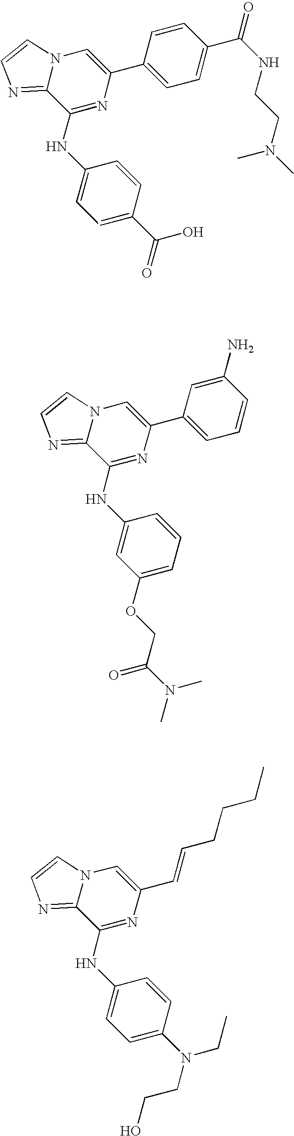

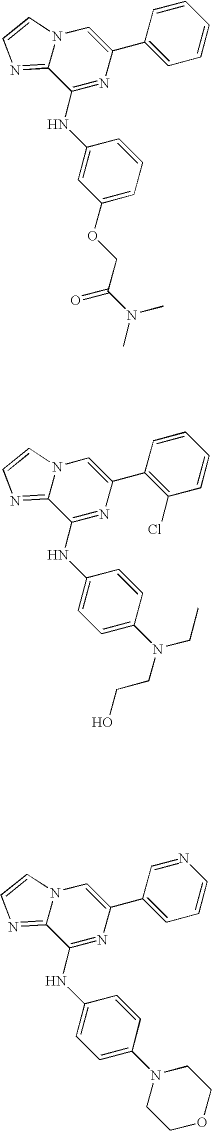

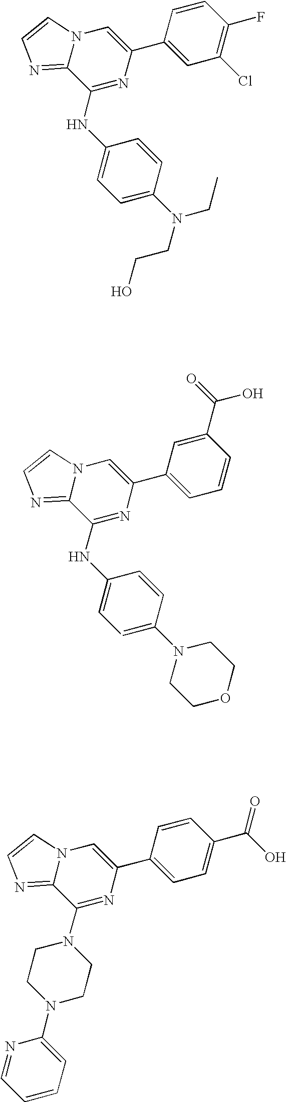

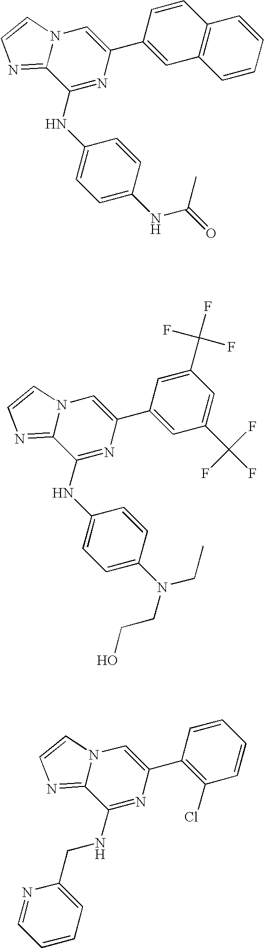

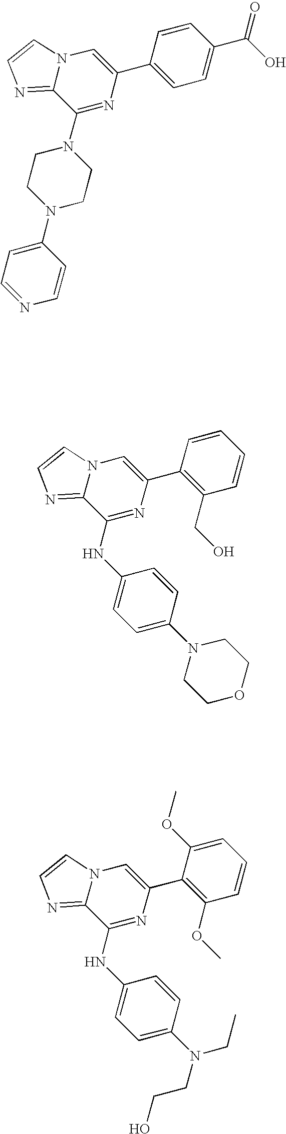

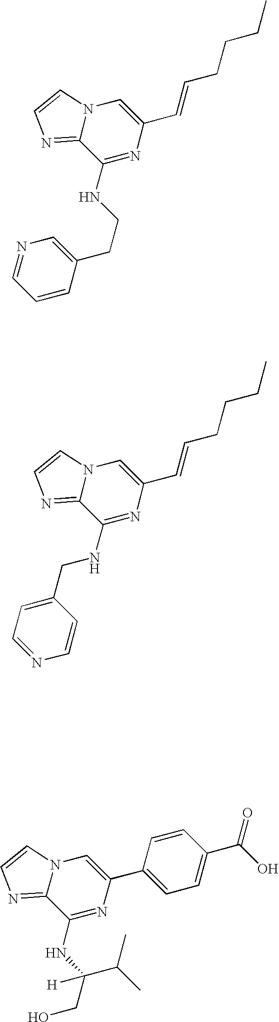

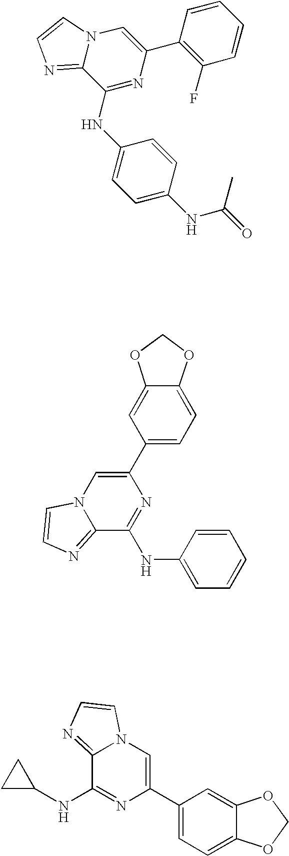

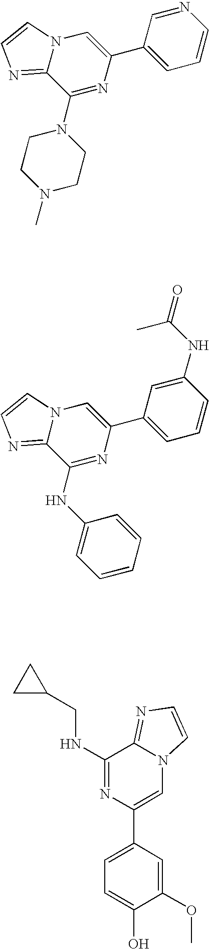

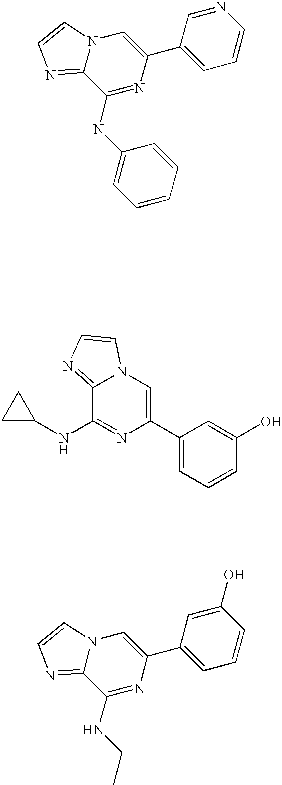

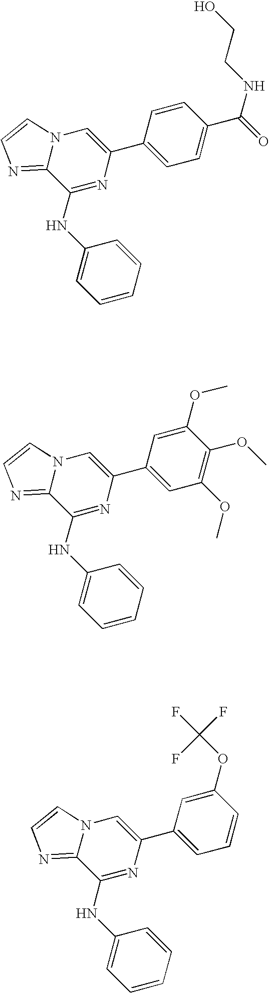

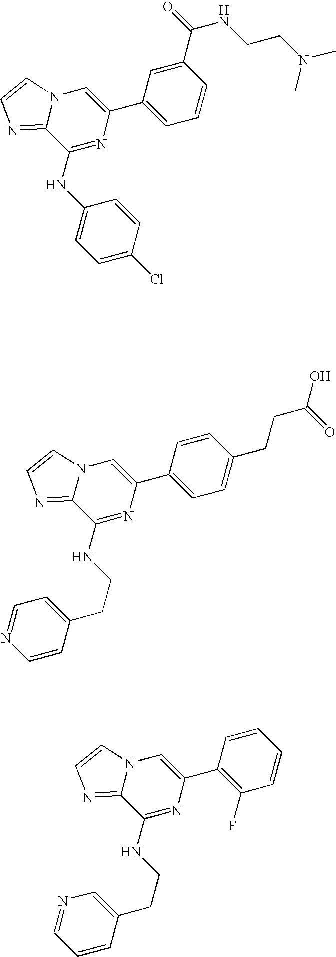

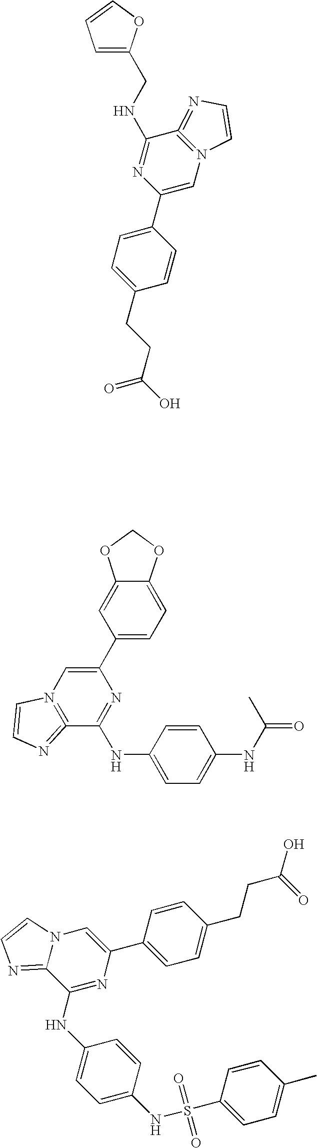

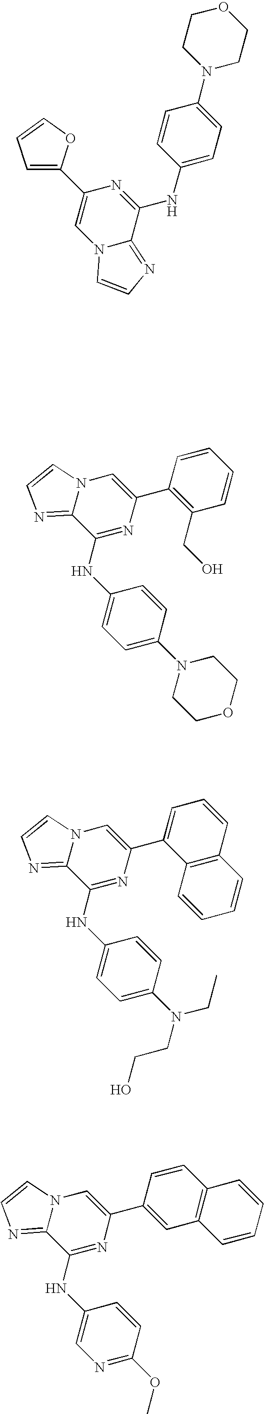

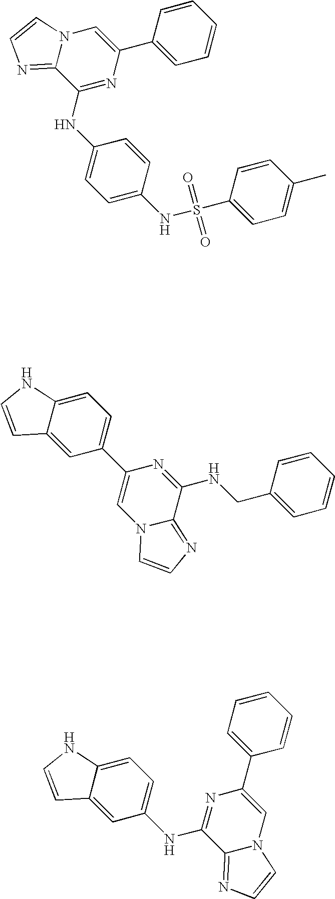

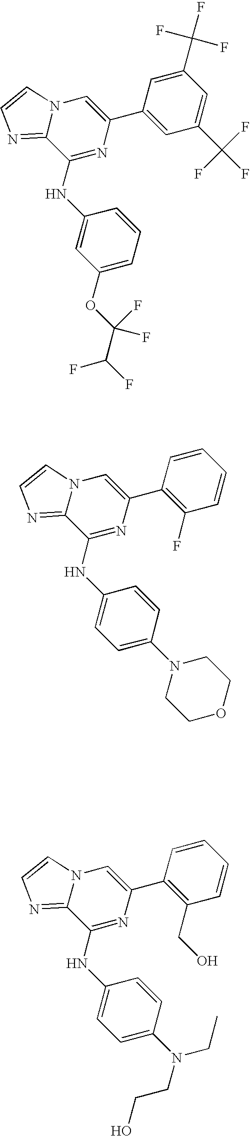

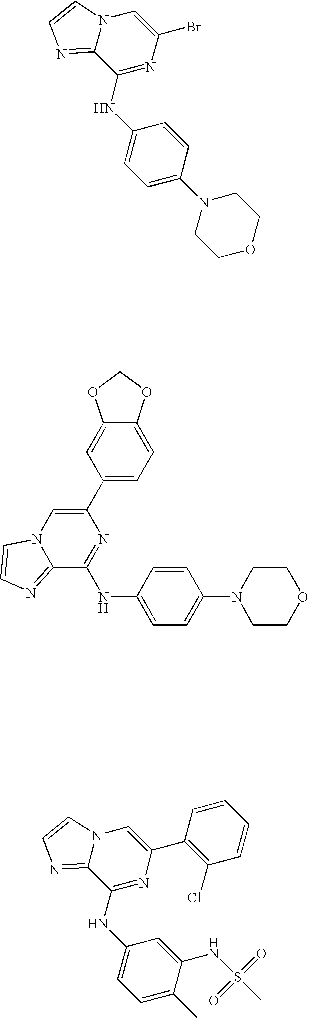

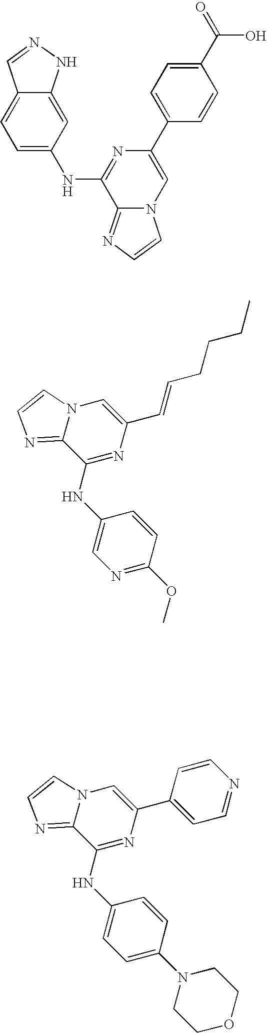

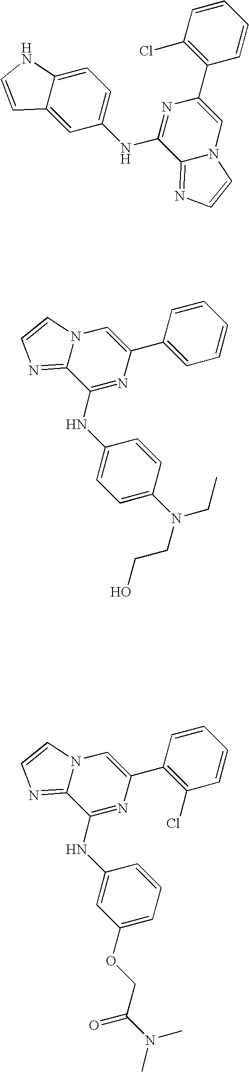

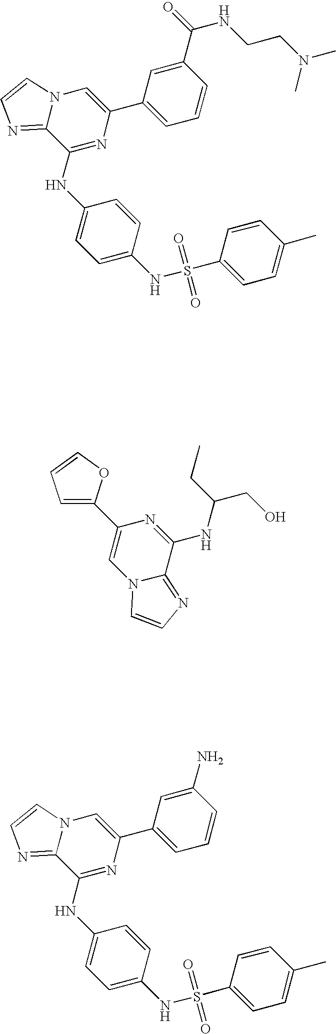

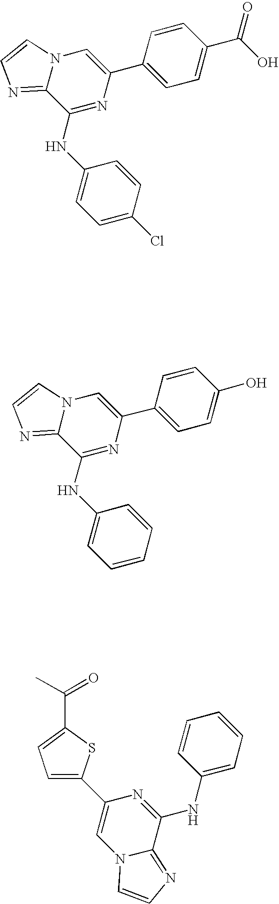

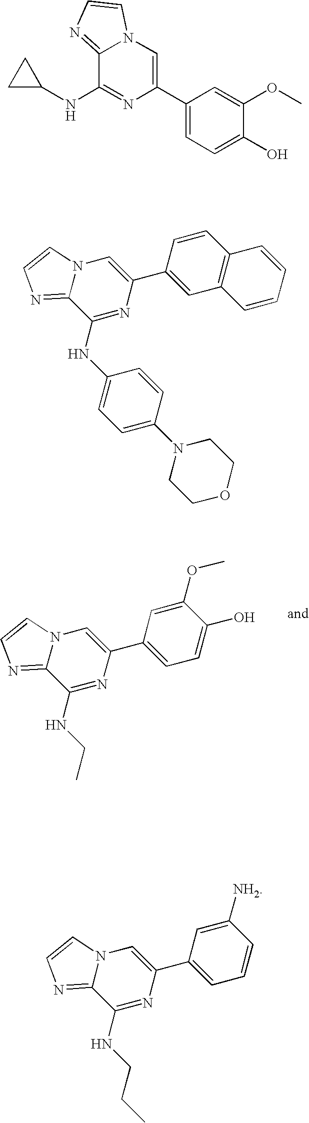

41. A method as claimed in claim 35 wherein the method is conducted in vivo.

42. A method as claimed in claim 35 wherein the JAK is JAK1.

43. A method as claimed in claim 35 wherein the JAK is JAK2.

44. A method as claimed in claim 35 wherein the JAK is JAK3.

45. A method as claimed in claim 35 wherein the JAK is TYK2.

46. A method of treating an individual suffering from a JAK-associated disease state, which comprises administering to the individual a composition comprising a pharmaceutically acceptable carrier and a compound of the general formula: 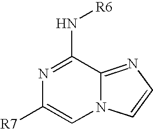
or pharmaceutically acceptable salts, hydrates, solvates, crystal forms or diastereomers thereof, wherein
R6 is C₁₋₁₀ alkyl, C₂₋₁₀ alkenyl, C₂₋₁₀ alkynyl, C₂₋₁₀ alkylaryl, aryl, or heterocycle, or R6 with N may form a substituted or unsubstituted heterocycle, wherein the alkyl, alkenyl, alkynyl, alkylaryl, aryl, and heterocycle, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl wherein heterocycle is a 5-7 membered ring and wherein the hetero atom is O, N or S;
R7 is C₁₋₁₀ alkyl, C₂₋₁₀ alkenyl, C₂₋₁₀ alkynyl, C₂₋₁₀ alkylaryl, aryl, halo, OH, or heterocycle, wherein the alkyl, alkenyl, alkynyl, alkylaryl, aryl, and heterocycle, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl wherein heterocycle is a 5-7 membered ring and wherein the hetero atom is O, N or S.

47. A method as claimed in claim 46 wherein the compound is of the general formula: 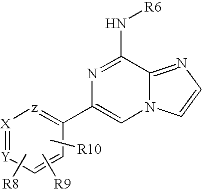
wherein one of X, Y or Z is nitrogen and the other two are carbon, or all three are carbon
R8, R9 and R10 are the same or different and are H, halo, OH, hydroxyamide, amino, hydroxyalkyl, aminoalkylamide, alkylamide, arylamide or alkoxy.

48. A method as claimed in claim 47 wherein R6 with N forms a heterocycle it is preferred that the heterocycle.

49. A method as claimed in claim 48 wherein the heterocycle includes two heteroatoms, preferably two nitrogen atoms.

50. A method as claimed in claim 47 wherein the compound is of the general formula: 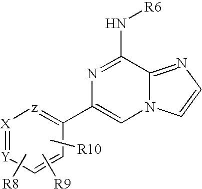
wherein:
R6 is C₂₋₁₀ alkylphenyl, phenyl, or heterocycle, wherein the alkyl, phenyl, and heterocycle, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular methoxy), heterocyclic alkyl wherein heterocycle is a 5-7 membered ring and wherein the hetero atom is O, N or S;
R3, R4 and R5 are the same or different and are H, halo, OH, hydroxyamide, amino, hydroxyalkyl, aminoalkylamide, alkylamide, arylamide or alkoxy.

51. A method as claimed in claim 47 wherein the compound is selected from the group consisting of:

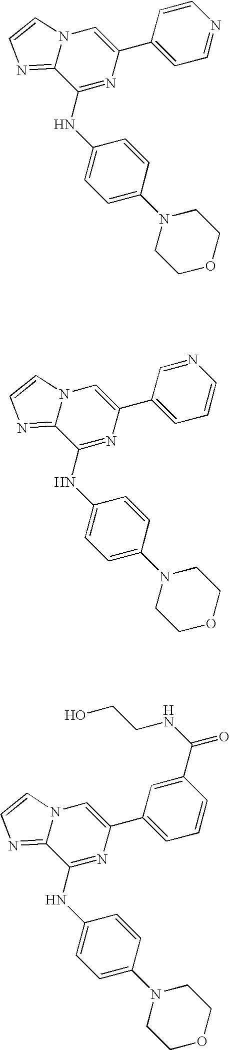

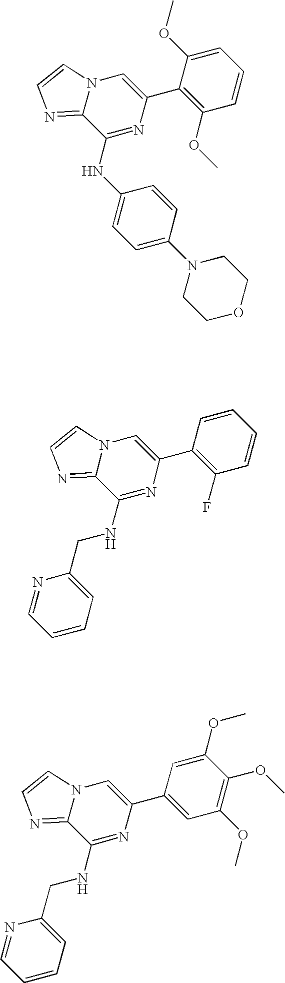

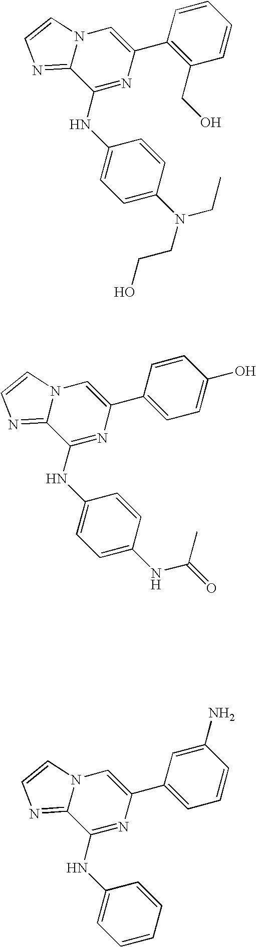

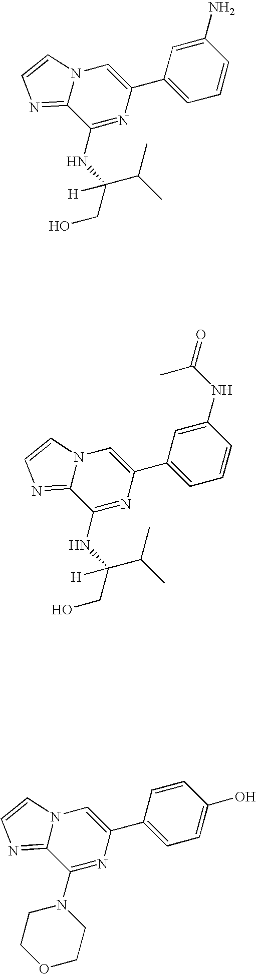

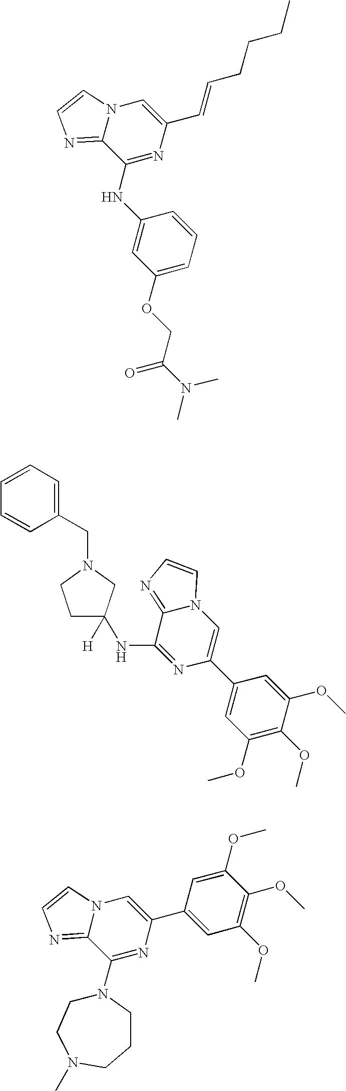

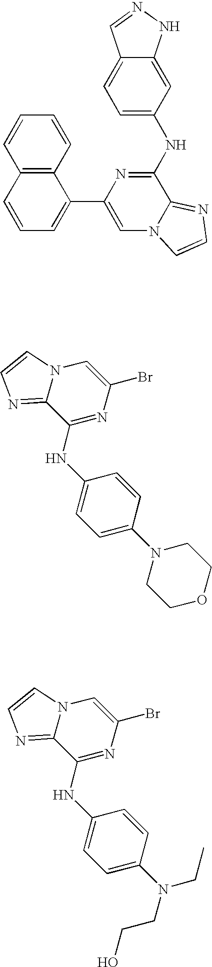

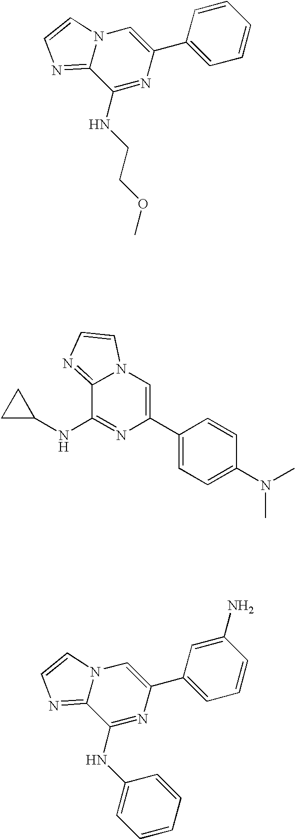

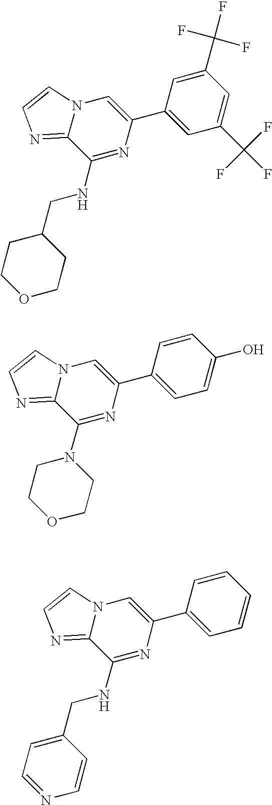

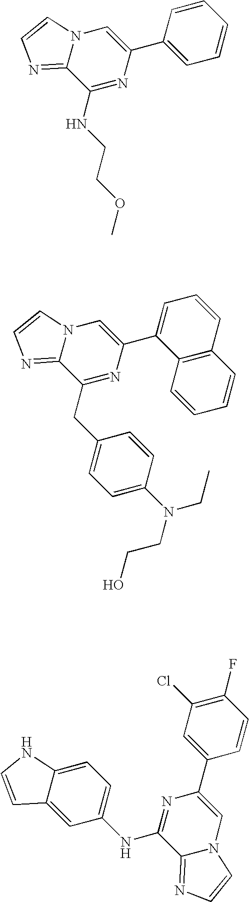

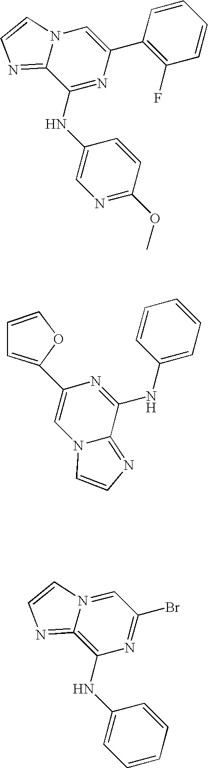

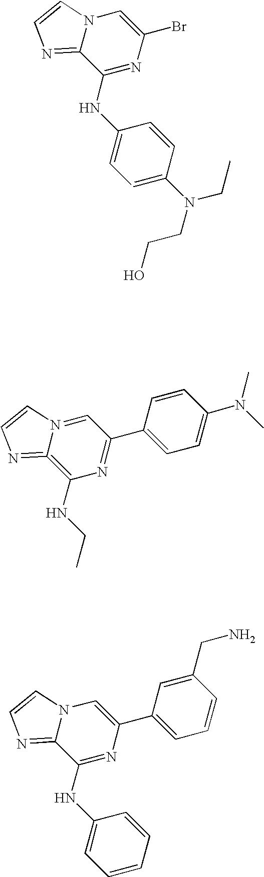

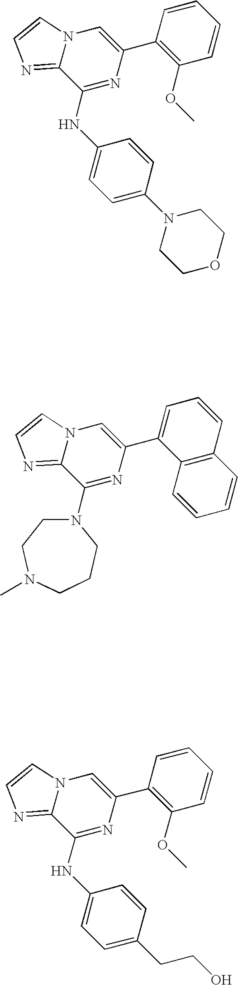

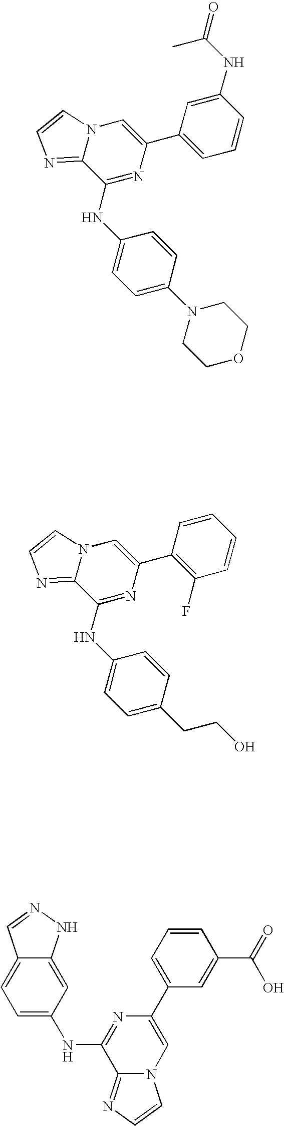

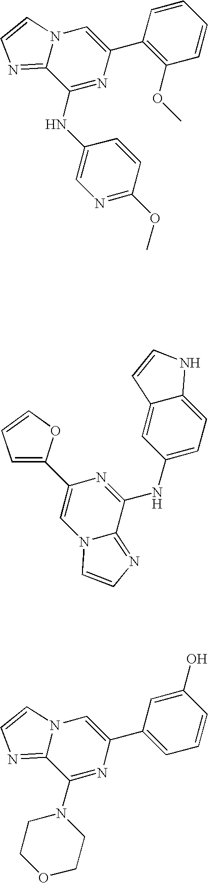

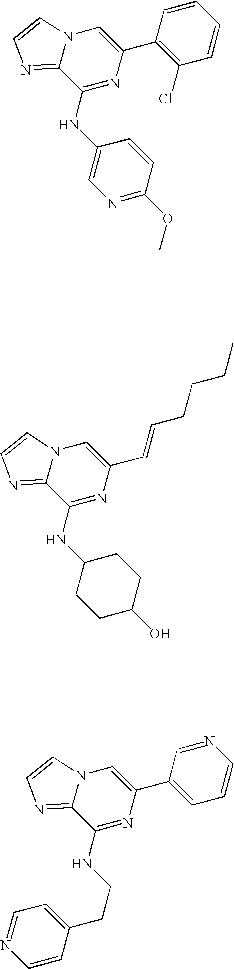

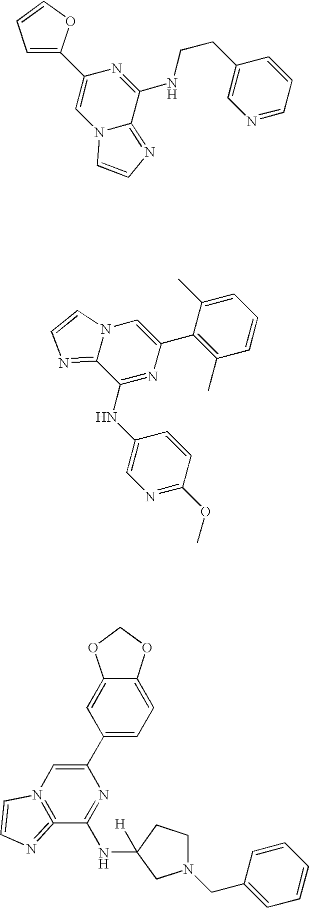

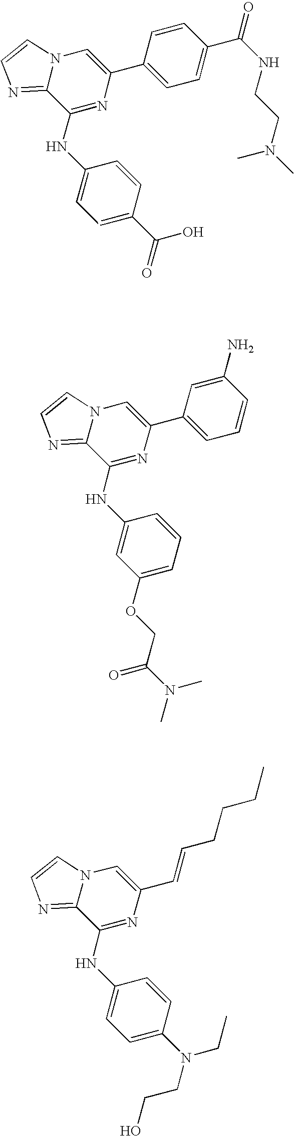

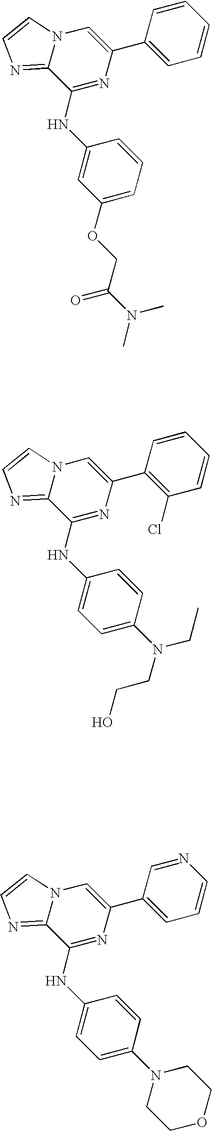

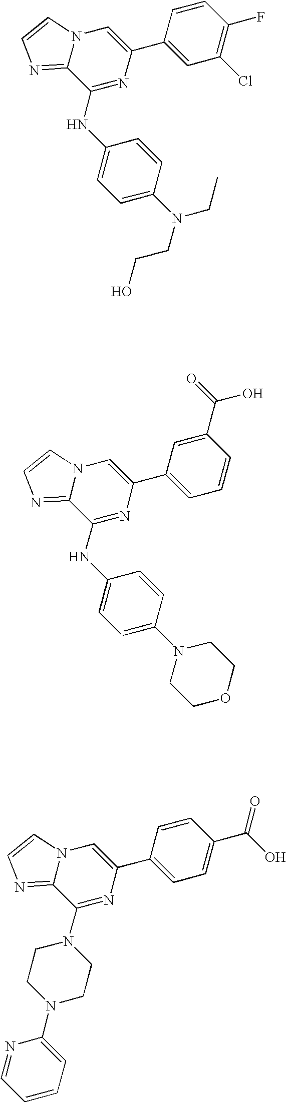

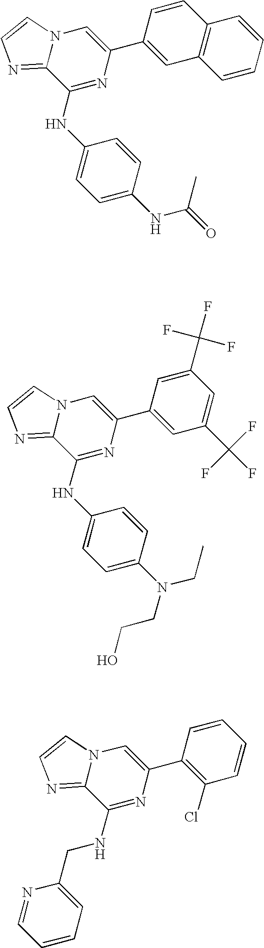

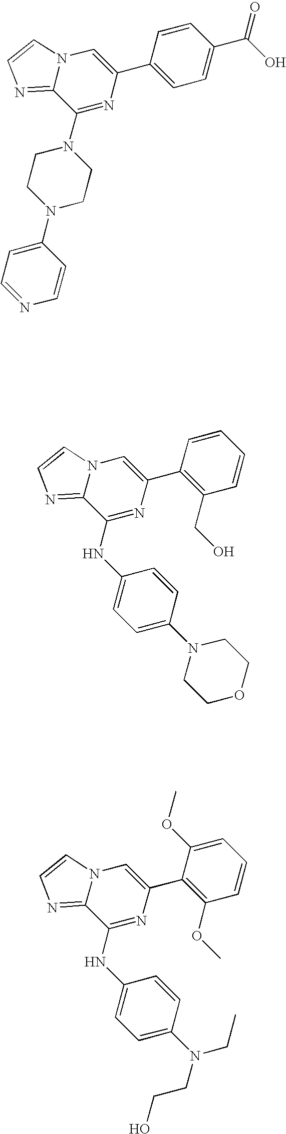

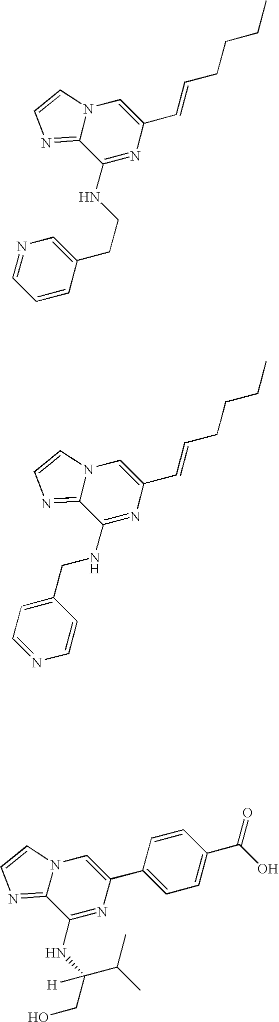

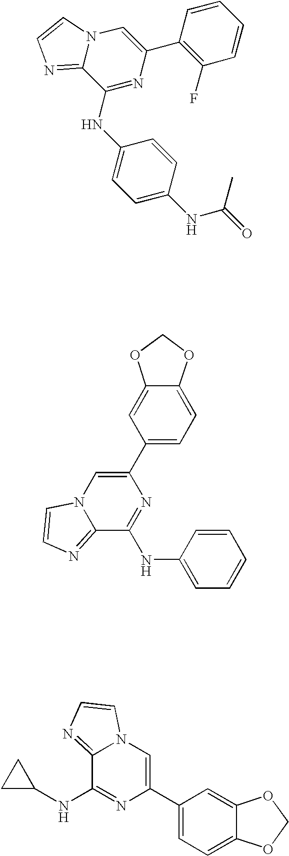

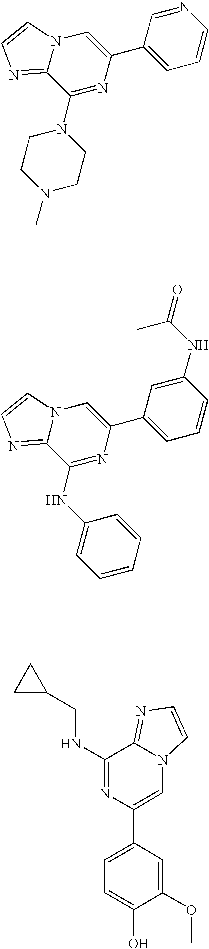

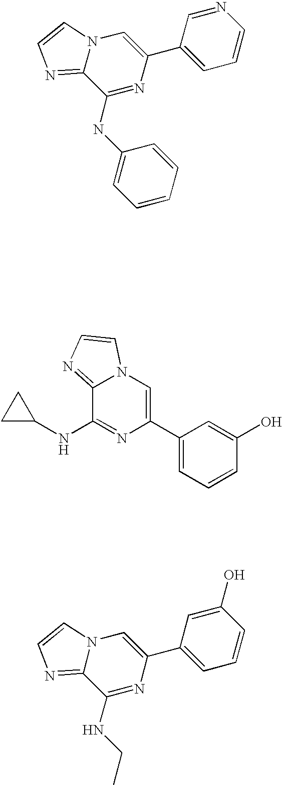

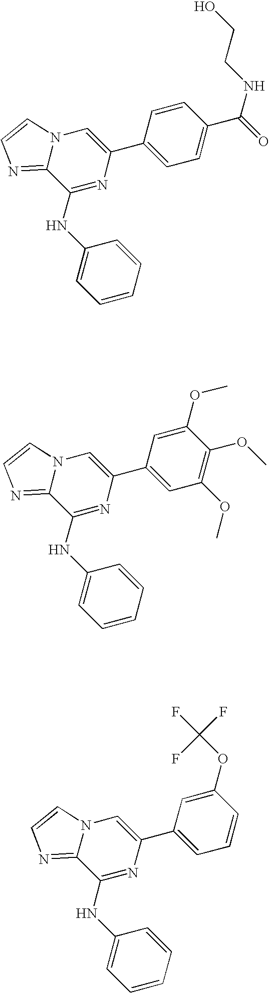

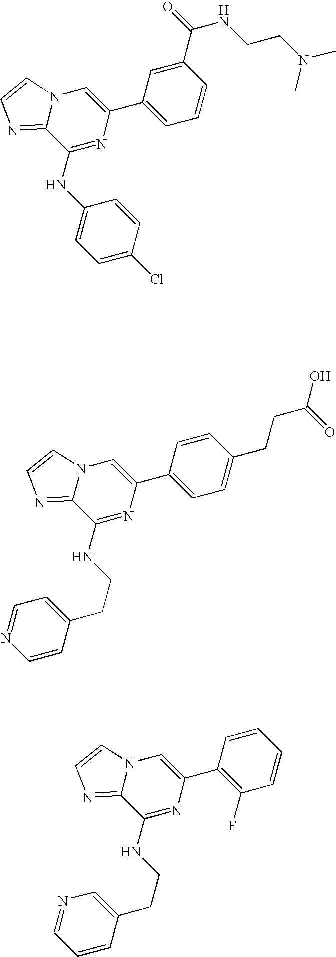

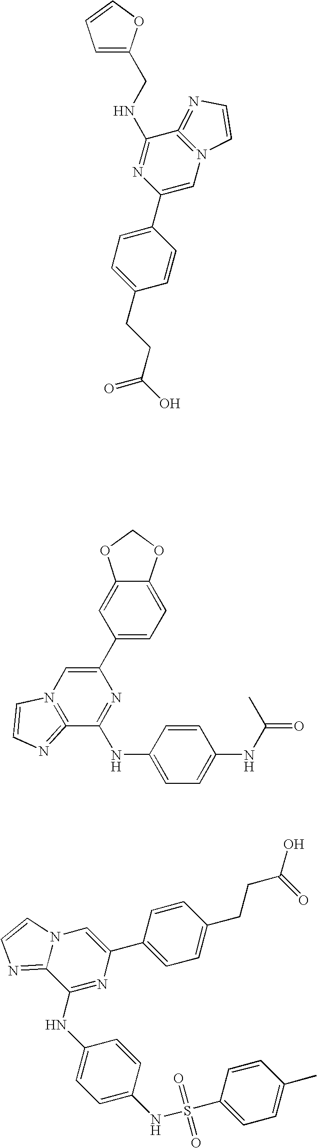

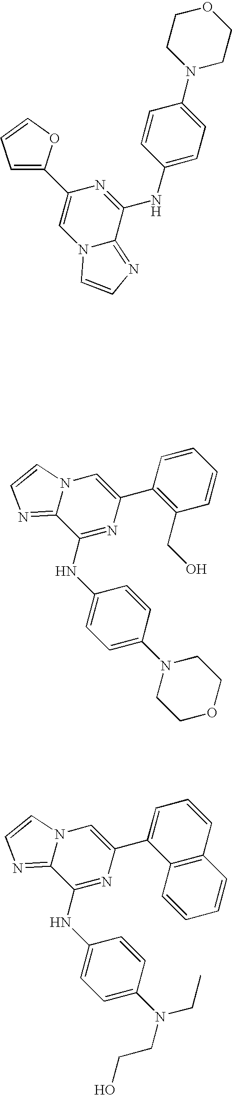

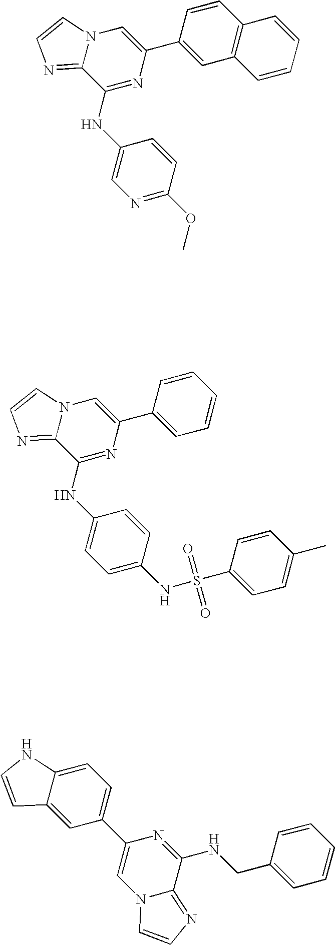

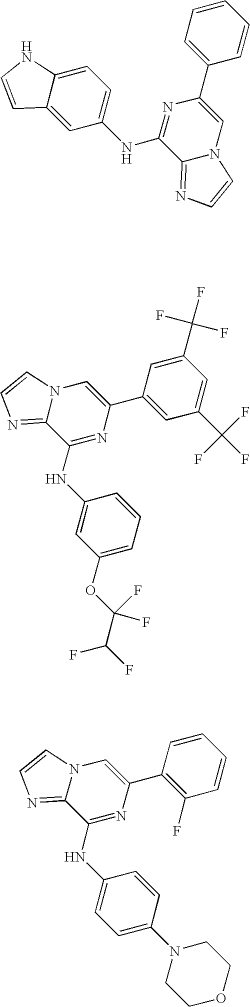

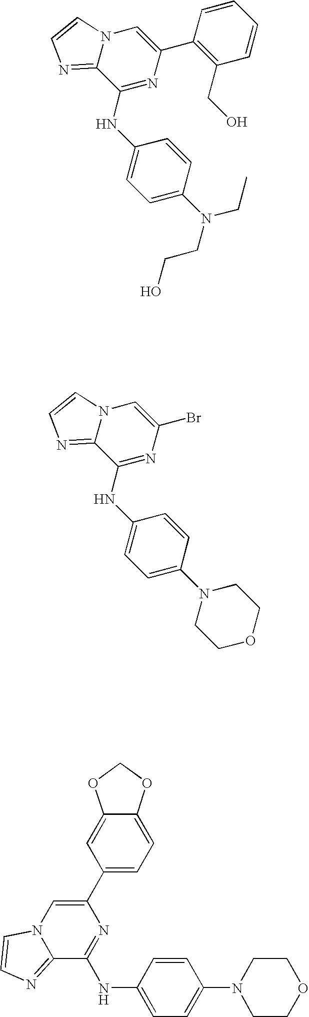

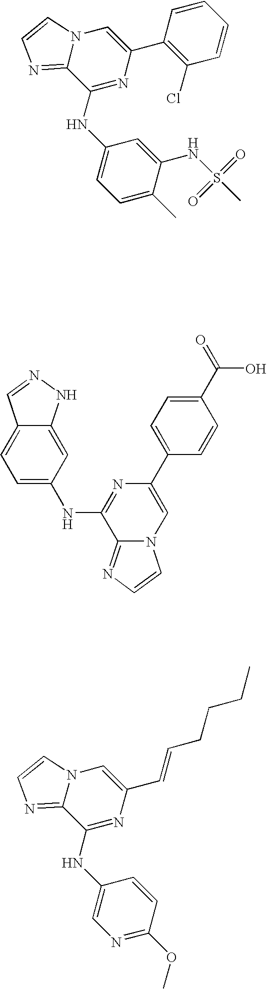

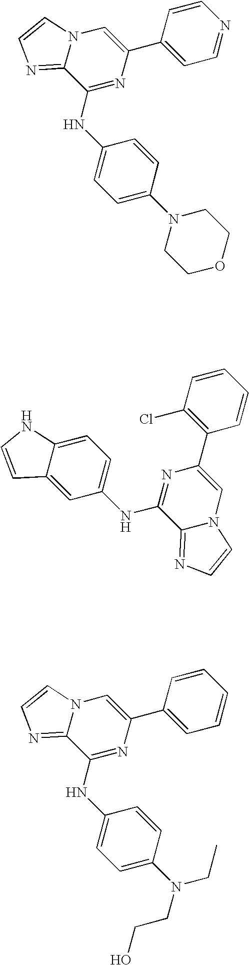

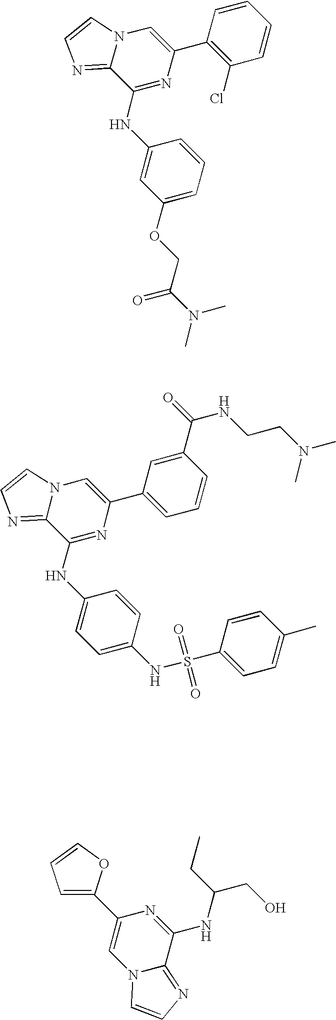

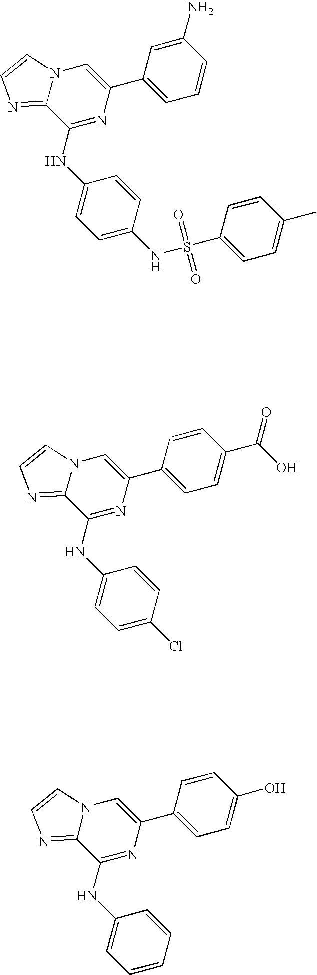

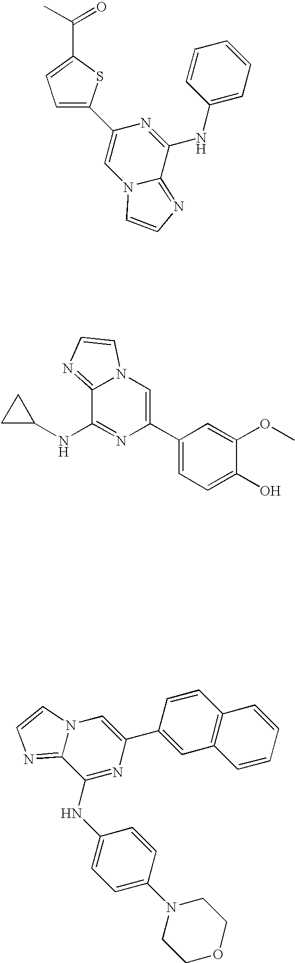

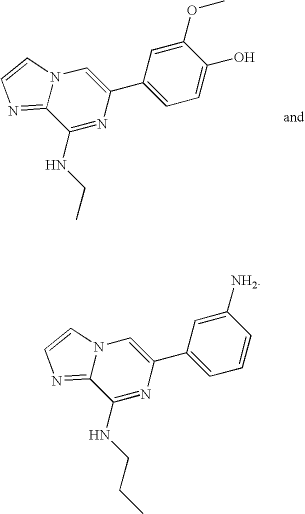

52. A method as claimed in claim 47 wherein the JAK-associated disease state involves JAK1.

53. A method as claimed in claim 47 wherein the JAK-associated disease state involves JAK2.

54. A method as claimed in claim 47 wherein the JAK-associated disease state involves JAK3.

55. A method as claimed in claim 47 wherein the JAK-associated disease state involves TYK2.

56. A method as claimed in claim 47 wherein the JAK-associated disease state is selected from the group consisting of an atopy, a cell mediated hypersensitivity, a rheumatic disease, an autoimmune disease, a viral disease, and a cancer.

57. A method as claimed in claim 56 wherein: the atopy is allergic asthma, atopic dermatitis (eczema), or allergic rhinitis; the cell mediated hypersensitivity is allergic contact dermatitis or hypersensitivity pneumonitis; the rheumatic disease is systemic lupus erythematosus (SLE), rheumatoid arthritis, juvenile arthritis, Sjögren's syndrome, scleroderma, polymyositis, ankylosing spondylitis, or psoriatic arthritis; the autoimmune disease is Type I diabetes, autoimmune thyroid disorders, or Alzheimer's disease; the viral disease is Epstein Barr virus (EBV), Hepatitis B, Hepatitis C, HIV, HTLV 1, Varicella-Zoster virus (VZV), or human papilloma virus (HPV); the cancer is leukemia, lymphoma or prostate cancer.

58-80. (canceled)


SEQUENCE LISTING (9 sequences)

SEQ ID NO: 1 - DNA, 30 bp, Artificial Sequence
  Primer
ccgctcgagactgaagtggaccccacacat

SEQ ID NO: 2 - DNA, 30 bp, Artificial Sequence
  Primer
cggggtaccttattttaaaagtgcttcaaa

SEQ ID NO: 3 - DNA, 31 bp, Artificial Sequence
  Primer
acgcgtcgacggtgcctttgaagaccgggat

SEQ ID NO: 4 - DNA, 34 bp, Artificial Sequence
  Primer
atagtttagcggccgctcagaatgaaggtcattt

SEQ ID NO: 5 - DNA, 30 bp, Artificial Sequence
  Primer
ccgctcgagtatgcctgccaagaccccacg

SEQ ID NO: 6 - DNA, 30 bp, Artificial Sequence
  Primer
cggggtaccctatgaaaaggacagggagtg

SEQ ID NO: 7 - DNA, 33 bp, Artificial Sequence
  Primer
ggagcactcgagatggtagcacacaaccaggtg

SEQ ID NO: 8 - DNA, 35 bp, Artificial Sequence
  Primer
ggagcaggaattccggcgctgccggtcaaatctgg

SEQ ID NO: 9 - protein, 17 aa, Artificial Sequence
  Synthetically engineered gastrin producing peptide
EGPWLEEEEEAYGWMDF